(19) United States
(12) Patent Application Publication
(10) Pub. No.: US 2025/0346946 A1
(43) Pub. Date: Nov. 13, 2025

(54) QUANTIFICATION OF CO-LOCALIZED TAG SEQUENCES USING ORTHOGONAL SEQUENCE ENCODING

(71) Applicant: Ultima Genomics, Inc., Fremont, CA (US)

(72) Inventors: Itai RUSINEK, Holon (IL); Zohar SHIPONY, Rehovot (IL); Omer BARAD, Mazkeret Batya (IL); Ariel HAIMOVICH, Menlo Park, CA (US)

(21) Appl. No.: 19/236,815

(22) Filed: Jun. 12, 2025

Related U.S. Application Data
(63) Continuation of application No. PCT/US2023/085241, filed on Dec. 20, 2023.
(60) Provisional application No. 63/476,892, filed on Dec. 22, 2022.

(51) Int. Cl. C12Q 1/6837; C12Q 1/6874; C12Q 1/6876

(52) U.S. Cl. CPC C12Q 1/6837; C12Q 1/6874; C12Q 1/6876

(57) ABSTRACT

Despite the advance of screening technology, omic-based studies with spatial resolution still requires laborious efforts, hampering the analysis of biology and disease. The present disclosure provides methods, systems, probes, and platforms that may be based on the use of flow-based sequencing to increase the throughput of analyte screening with spatial resolution.

### CROSS-REFERENCE

This application is a continuation of International Application No. PCT/US2023/085241, filed Dec. 20, 2023, which claims the benefit of U.S. Provisional Patent App. No. 63/476,892, filed Dec. 22, 2022, which are entirely incorporated herein by reference.

### STATEMENT REGARDING SEQUENCE LISTING

This application contains a Sequence Listing, which has been submitted electronically in xml format and is hereby incorporated by reference in its entirety. Said xml copy, created on Jul. 11, 2025, is named SeqList-368893-40701.xml and is 2,830 bytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts. For example, sample processing for spatial multi-omics may include the analysis of messenger RNA (mRNA) transcripts, proteins, and/or genomic DNA (gDNA).

### SUMMARY

Despite the prevalence of biological sample processing systems and methods, such systems and methods may have low efficiency that can be time-intensive and wasteful of valuable resources, such as reagents. The processing of a biological sample may include a spatial analysis of analytes (e.g., RNA transcripts or proteins) in single cells. Recognized herein is a need for improved methods and systems for sample processing and multiplexed measurements for spatial multi-omics.

Bulk assays, e.g., DNA sequencing, RNA sequencing, and mass spectrometry, may detect DNA, RNA, proteins, and the like in a substrate. These approaches are useful for studying differences in bulk populations. However, they remain unable to provide details on individual cell phenotypes and cell-to-cell variability.

Single-cell analysis techniques, e.g., flow cytometry and single-cell sequencing, provide a higher resolution picture of a sample. For example, RNA and/or protein expression may be measured in single cells to catalogue differences at the single-cell level. However, such analysis techniques may not characterize the spatial organization and interactions between cells.

Spatial biology includes profiling RNA and protein expression in a spatially resolved manner, and thus provides methods of characterizing single cells and tissues in two- and three-dimensions. In this way, it is possible to understand how cells organize and interact within tissues in the context of disease and therapy response. For example, spatial multi-omics may reveal such spatial relationships and interactions by imaging whole tissue sections at single-cell resolution, and allow the characterization of analyte abundance, cell types and functional states, and cellular organization and interactions.

Spatial multi-omics may include (1) transcriptomics, where mRNA transcripts are detected, such as by probe hybridization; (2) proteomics, where proteins are detected, such as by binding an antibody and measuring its concentration, although potentially antibodies for molecules other than proteins may be included as well (e.g., for metabolomics); and/or (3) genomics, where gDNA is detected, such as to detect copy number alteration or specific structural or short variants. Spatial multi-omics may identify, qualify, and/or quantify different analytes within any of these fields.

Spatial transcriptomics is an evolving technique that quantifies transcriptomes from tissue sections and allows analysis of a cell state while retaining spatial context. However, current spatial transcriptomics approaches may provide high levels of multiplexing at the cost of single-cell resolution, relying on region-of-interest or spot-based capture methods. For example, existing methods may rely on oligonucleotide hybridization to an RNA transcript, oligonucleotides attached to a binding antibody, or aptamers. Detection may be accomplished by fluorescence of an attached residue or various amplification schemes where multiple fluorescent residues bind to a single hybridized probe. Alternatively, fluorescence may be activated by multiple adjacent probes linked by a molecular interaction (e.g., FRET) to increase specificity.

Spatial proteomics is the large-scale analysis of proteins and their localization and dynamics within tissue. Current imaging-based spatial proteomics methods allow quantitative and spatial analysis of protein markers across a whole tissue section at single-cell resolution.

The ability to profile hundreds to thousands of transcripts makes single-cell, spatial transcriptomics a powerful tool for unbiased discovery and highly complementary to spatial proteomics. Such a spatial multi-omics approach that integrates single-cell spatial proteomic data with single-cell spatial transcriptomic data, may provide more detailed insights into tissue biology and the discovery of novel biomarker signatures. Examples of samples suitable for spatial multi-omics may include fresh frozen tissues, fixed tissues, and formalin-fixed, paraffin-embedded (FFPE) tissue.

Spatial multi-omics may be used in conjunction with next-generation sequencing (NGS). In classical applications of NGS, a high accuracy in the determination of sequences may be necessary to differentiate small changes and determine a specimen's sequence with high certainty. In many high throughput biological assays, the goal of the assay may not be to determine unknown sequences with high accuracy, but to identify and quantify sets of previously known sequences in a sample. For example, RNA-seq can count different transcripts in a cell or in bulk. For other applications, such as Olink™ proteomics using Proximity Extension Assay technology, sequencing requirements are lower still, as sequences are determined from a previously known set of 100s-1000s of synthetic sequences. However, currently these assays are generally carried out as standard NGS assays, which may yield redundant information and lower throughput.

Flow-based sequencing allows identification of different oligonucleotides, from sequential optical signals obtained in specific flows rather than from a full determination of its sequence. These oligonucleotides may be attached to, or be part of, a probe for a specific transcript, protein, or other target analyte. Oligonucleotide probes can therefore be defined or encoded in so-called flow space by their flow-space sequence, the sequence of integers that are measured for them in a specific flow order. Synthetic sets of sequences can be designed for a set of probes so that they are “temporally” separated in flow space. More generally, a set of sequences can be designed for a set of probes so that all the sequences are orthogonal to each other in flow space, and, in specific cases, resolution may be better than binary (zero vs non-zero). As a simple example, the ‘base’ sequences ACT and AGT under the flow order TGCA yield flow-space sequences of [1 1 0 1] and [1 0 1 1] respectively, so that the 2nd flow would be nonzero for the former only and the 3rd for the latter only.

This flow-based encoding allows for applications based on flow-based sequencing, where multiple colocalized sequences can be measured simultaneously from a single (or local) point in space, for example from one polyclonal bead, bridge-amplified colony, DNA nanoball, other colonies, or possibly single molecules. Such applications can include relative quantification of synthetic sequences attached to antibodies, aptamers or other affinity agents for the quantification of targets bound to biological entities of interest (e.g., cell surface markers); RNA-seq combined with isoform quantification by binding tags to various exons; and targeted single cell multi-omics by bin.

The present disclosure provides methods and systems for multiplexed measurements of multi-omics. The systems and methods may use oligonucleotides that are either attached to or part of a probe for a specific transcript, protein, or other target or analyte type. A probe may include a flow-based code domain comprising a nucleic acid sequence encoding a flow-space sequence, a primer binding site, and a target-related domain. In some cases, the flow-space sequence may be configured to generate a flowgram unique to the probe amongst a plurality of probes during flow-based sequencing, wherein the flowgram comprises a set of relative intensity values generated during the flow-based sequencing.

In particular, hundreds or more of probes may be designed that are specific to different targets and contain one or more primer binding sites. All the probes may be bound together to the sample of interest, before or after it is mounted the samples of interest to a silicon or glass wafer, slide, or another surface. Interrogation of the identity of the bound probes may be done using a DNA polymerase synthesizing a combination of natural and fluorescently-labelled nucleotides on the probe's reverse strand, as is done in flow-based sequencing, except that the identity of the probe is deduced from the optical signal in a few specific flows rather than from a full determination of its sequence. The nature of the signal in flow-based sequencing may be used for the multiplexing of hundreds of probes or more, by orthogonal encoding in so-called flow-space of the sequences attached to the probes. Each target can be encoded either in one unique flow where all the rest of the oligonucleotides yield no signal (or a baseline signal of a 0-mer), or combinatorically encoded in multiple flows, potentially along with multiple additional transcripts. Each target may have up to 10s of different probes, increasing signal magnitude and specificity. Additionally, higher multiplexing levels may be obtained by combining this flow-space encoding scheme with multi-channel encoding with different fluorophores.

The basic design of the probe may be composed of 3 domains:
(1) A target-related domain. This domain may either binds the target directly or may be attached to another molecule binding the target. For transcriptomics or genomics, the target binding domain may directly hybridize to the target RNA or DNA molecule. For protein or other antibody-binding-based targets, the target binding domain may be attached to the antibody, or may be an aptamer.
(2) A primer binding site, to which the sequencing primer is configured to be hybridized; and
(3) A flow-based code domain, the flow-based quasi-sequencing signal of which may be used to interrogate the identity of the probe.

In one aspect the present disclosure provides a probe comprising: a. a flow-based code domain comprising a nucleic acid sequence encoding a flow-space sequence; b. a first primer binding site; and c. a first target-related domain.

In some embodiments, the flow-based code domain is configured to generate a flowgram unique to the probe amongst a plurality of probes during flow-based sequencing, wherein the flowgram comprises a set of relative intensity values generated during the flow-based sequencing.

In some embodiments, the flow-space sequence comprises a key flow position every (3n−1)^{th} and (3n)^{th} flow positions, wherein n is a positive integer.

In some embodiments, the flow-based code domain is positioned 5′ to the first primer binding site, and the first primer binding site is positioned 5′ to the first target-related domain.

In some embodiments, the first target-related domain comprises an oligonucleotide, an aptamer, an antibody or binding fragment thereof, or a combination thereof.

In some embodiments, the first target-related domain is configured to bind to an analyte. In some embodiments, the analyte comprises an antibody or binding fragment thereof, an oligonucleotide, an RNA transcript, a protein, a polypeptide, a metabolite, or genomic DNA.

In some embodiments, the probe further comprises a first PCR primer binding site positioned 5′ to the flow-based code domain or 3′ to the first target-related domain.

In some embodiments, the probe further comprises a bead adapter sequence 3′ to the first target-related domain.

In some embodiments, the probe comprises a first strand and a second strand, wherein the first strand comprises the first target-related domain and the first primer binding site, wherein the second strand comprises a sequencing primer hybridized to the first primer binding site. In some embodiments, the second strand further comprises a second target-related domain. In some embodiments, the second strand further comprises a linker between the sequencing primer and the second target-related domain. In some embodiments, the linker is positioned 5′ to the sequencing primer and the second target-related domain is positioned 5′ to the linker. In some embodiments, the second target-related domain is configured to bind to an analyte. In some embodiments, the analyte comprises an antibody or binding fragment thereof, an oligonucleotide, an RNA transcript, a protein, a polypeptide, a metabolite, or genomic DNA. In some embodiments, the first target-related domain and the second target-related domain are configured to bind to two different targets. In some embodiments, the two different targets are two different locations on a same molecule.

In another aspect, provided is a two-part probe, comprising: a 5′ probe and a first PCR primer binding site positioned 5′ to the flow-based code domain; and a 3′ probe comprising a ligation target-related domain and a second PCR binding site.

In some embodiments, the first target-related domain is configured to bind to an oligonucleotide.

In some embodiments, the ligation target-related domain is configured to bind to an oligonucleotide. In some embodiments, the first target-related domain and the ligation target-related domain are configured to bind to adjacent locations on the oligonucleotide.

In some embodiments, the probe is bound to a target, wherein a tissue slice comprises the target.

In some embodiments, the probe is bound to a target that is immobilized on a substrate. In some embodiments, the substrate is a Z-slice, a slide, a silicon wafer, or a glass wafer.

In some embodiments, the first target-related domain binds to a target via a binding agent. In some embodiments, the binding agent comprises an oligonucleotide-conjugated antibody.

In another aspect, provided is a two-part probe, comprising: a first probe part comprising the probe of any one of the aforementioned embodiments and further comprising a first annealing domain positioned 5′ to the flow-based coding domain, wherein the first target-related domain comprises a first antibody; and a second probe part comprising a second target-related domain comprising a second antibody, a second primer binding site, and a second annealing domain, wherein the second annealing domain is configured to be a reverse complement of the first annealing domain; wherein the first antibody and the second antibody bind to two different targets, wherein the two different targets are different locations on a same molecule.

In some embodiments, the second probe part further comprises a bead-binding domain.

In some embodiments, at least two probes each encode a unique flow-space sequence.

In another aspect, provided is a method, comprising: a. binding a probe to a target, the probe comprising: a first flow-based code domain comprising a nucleic acid sequence encoding a flow-space sequence; a first primer binding site; and a first target-related domain configured to bind to the target; b. hybridizing a sequencing primer to the first primer binding site of the probe; and c. sequencing at least a portion of the flow-based sequence using flow-based sequencing to generate a flowgram unique to the probe amongst a plurality of probes during flow-based sequencing, wherein the flowgram comprises a set of relative intensity values generated during the flow-based sequencing.

In some embodiments, the method further comprises using the flowgram to determine an identity of the target.

In some embodiments, the target is an analyte, an antibody or fragment thereof, an oligonucleotide, an RNA transcript, a protein, a polypeptide, a metabolite, or genomic DNA.

In some embodiments, the method further comprises immobilizing the target on a substrate prior to, during, or subsequent to binding to the probe. In some embodiments, the method further comprises using the flowgram to determine a location and/or distribution of the target on the substrate. In some embodiments, the substrate is a Z-slice, a slide, a silicon wafer, or a glass wafer.

In some embodiments, the substrate further comprises a capture oligonucleotide, and the method further comprises: releasing the probe from the target; and binding the probe to the capture oligonucleotide. In some embodiments, binding the probe to the capture oligonucleotide is facilitated by electrophoresis, a magnetic field, or a combination thereof.

In some embodiments, the target is immobilized on a capture bead.

In some embodiments, the probe is immobilized on a capture bead.

In some embodiments, the probe comprises more than one flow-based code domain and more than one primer binding site.

In some embodiments, the probe comprises the probe or two-part probe of any one of the aforementioned embodiments.

In another aspect, provided is a method comprising: a. binding a first probe to a first target, wherein the first probe comprises: a first flow-based code domain comprising a first nucleic acid sequence encoding a first flow-space sequence; a first primer binding site; and a first target-related domain that is configured to bind to the first target; b. binding a second probe to a second target, wherein the second probe comprises: a second flow-based code domain comprising a second nucleic acid sequence encoding a second flow-space sequence; a second primer binding site; and a second target-related domain that is configured to bind to the second target; c. hybridizing a first sequencing primer and a second sequencing primer to the first primer binding site and the second primer binding site, respectively; and d. sequencing a portion of the first flow-space sequence and a portion of the second flow-space sequence using flow-based sequencing to generate a first flowgram and a second flowgram, respectively, wherein the first flowgram and the second flowgram are unique to each other.

In some embodiments, the first primer binding site and the second primer binding site comprise an identical sequence.

In some embodiments, the first primer binding site and the second primer binding site comprise different sequences.

In some embodiments, the method further comprises using the first flowgram and/or the second flowgram to determine an identity of the first target and/or the second target, respectively.

In some embodiments, the first target and/or the second target is an analyte. In some embodiments, the analyte comprises an antibody or binding fragment thereof, an oligonucleotide, an RNA transcript, a protein, a polypeptide, a metabolite, or genomic DNA. In some embodiments, the method further comprises immobilizing the first target and/or second target on a substrate prior to, during, or subsequent to binding the first probe and/or second probe.

In some embodiments, the method further comprises using the first flowgram and the second flowgram to determine the location and/or distribution of at least the first target and/or second target, respectively, on the substrate. In some embodiments, the substrate is a Z-slice, a slide, a silicon wafer, or a glass wafer.

In some embodiments, the first probe and/or the second probe comprises multiple flow-based code domains and multiple primer binding sites.

In some embodiments, the first probe and/or the second probe comprise the probe or two-part probe of any one of claims 1-28.

In some embodiments, the first target and/or the second target are from a single cell.

In another aspect, provided is a method comprising: a. immobilizing a first probe and a second probe on a substrate, wherein: the first probe comprises: a first flow-based code domain comprising a first nucleic acid sequence encoding a first flow-space sequence; a first target-related domain that binds to a first target immobilized on the substrate; and a first primer binding site; and the second probe comprises: a second flow-based code domain comprising a second nucleic acid sequence encoding a second flow-space sequence; a second target-related domain that binds to a second target immobilized on the substrate; and a second primer binding site; b. hybridizing a first sequencing primer and a second sequencing primer to the first primer binding site and the second primer binding site, respectively; and c. sequencing a portion of the first flow-based code domain and a portion of the second flow-based code domain using flow-based sequencing to generate a first flowgram and a second flowgram, respectively, wherein the first flowgram and the second flowgram are unique to each other.

In some embodiments, the first primer binding site and the second primer binding site comprise an identical sequence. In some embodiments, the first primer binding site and the second primer binding site comprise different sequences. In some embodiments, the method further comprises using the first flowgram and the second flowgram to determine the location and/or distribution of at least the first target and/or second target, respectively, on the substrate.

In some embodiments, the substrate is a Z-slice, a slide, a silicon wafer, or a glass wafer.

In some embodiments, the first probe and/or the second probe comprises multiple flow-based code domains and multiple primer binding sites.

In some embodiments, the first probe and/or the second probe comprise the probe or two-part probe of any one of the embodiments above.

In some embodiments, the first target and/or the second target are from a single cell.

In another aspect, provided is a method comprising: a. binding a plurality of probes to a plurality of targets, wherein each probe comprises: a flow-based code domain comprising a nucleic acid sequence encoding a flow-space sequence; a primer binding site; and a target-related domain; wherein a plurality of target-related domains binds to the plurality of targets; b. hybridizing a plurality of sequencing primers to a plurality of primer binding sites; and c. sequencing a plurality of flow-space sequences of the plurality of probes using flow-based sequencing to generate a plurality of flowgrams, wherein each unique flowgram corresponds to a probe bound to a unique target.

In some embodiments, the method further comprises using the plurality of flowgrams to determine an identity of the plurality of targets.

In some embodiments, the plurality of primer binding sites comprises an identical sequence. In some embodiments, the plurality of primer binding sites comprises different sequences.

In some embodiments, the method further comprises immobilizing the plurality of targets on a substrate prior to, during, or subsequent to binding the plurality of probes.

In some embodiments, the method further comprises using the first flowgram and the second flowgram to determine the location and/or distribution of at least the first target and/or second target, respectively, on the substrate.

In some embodiments, the substrate is a Z-slice, a slide, a silicon wafer, or a glass wafer.

In some embodiments, the plurality of probes comprises a probe comprising multiple flow-based code domains and multiple primer binding sites.

In some embodiments, the plurality of probes comprises the probe or two-part probe of any one of the above embodiments.

In some embodiments, the plurality of targets is from a single cell.

In another aspect, provided is a system comprising: a sequencing platform configured to perform flow-based sequencing; a plurality of probes, wherein each unique probe comprises a flow-based code domain comprising a nucleic acid sequence encoding a flow-space sequence, a target-related domain and a primer binding site; and a substrate comprising a target, wherein the target-related domain is bound to the target.

In some embodiments, the plurality of probes comprises the probe or two-part probe of any one of the above embodiments.

In another aspect, provided is a kit comprising: a plurality of probes, each probe comprising: a flow-based code domain comprising a nucleic acid sequence encoding a flow-space sequence; a first primer binding site; and a target-related domain; and instructions for use according to any one of the methods described in the embodiments above.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing, as disclosed herein.

FIG. 2 illustrates examples of individually addressable locations distributed on substrates, as described herein.

FIGS. 3A, 3B, 3C, 3D, 3E, 3F, and 3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule, as described herein.

FIGS. 7A, 7B, and 7C illustrate multiplexed stations in a sequencing system, as described herein.

FIG. 8 illustrates an exemplary flow-based sequencing method for generating sequencing data, as described herein.

FIGS. 9A-9B illustrate exemplary detected signals and corresponding determined sequence after five exemplary flow cycles are performed, as described herein.

FIGS. 10A, 10B, and 10C illustrate exemplary probes that include a flow-based code domain, as described herein.

FIGS. 11A, 11B, and 11C illustrate other exemplary probes that include a flow-based code domain, as described herein. FIG. 11A illustrates a two-part probe design that can be used for in-situ sequencing. FIG. 11B illustrates a two-part probe design that can be used in a splint ligation scheme. FIG. 11C illustrates a two-part probe design that can be used in a bispecific antibody binding scheme.

FIG. 12 illustrates a computer system that is programmed or otherwise configured to implement methods provided herein.

FIG. 13 illustrates an exemplary in-situ workflow using the probes of the present disclosure, as described herein.

FIG. 14 illustrates an exemplary probe extraction workflow using the probes of the present disclosure, as described herein.

FIGS. 15A, 15B, and 15C illustrate exemplary polyclonal bead-based workflows using the probes of the present disclosure, as described herein.

FIG. 16 illustrates an alternative exemplary polyclonal bead-based workflow using the probes of the present disclosure, as described herein.

FIG. 17 illustrates an exemplary workflow for single cell mRNA-isoform sequencing using the probes of the present disclosure, as described herein.

FIG. 18 illustrates an exemplary workflow for targeted ultra-high-throughput single cell transcriptomics using the probes of the present disclosure, as described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(SEQ ID NO: 1)
(e.g., [A T G C A T G C A T G A T G A T G A T
G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order, and can be expressed in units of flow cycles. For example, where [A T G C] is identified as a 1^{st} flow cycle, and [A T G] is identified as a 2^{nd} flow cycle, the flow order of [A T G C A T G C A T G A T G A T G A T G C A T G C] (SEQ ID NO:1) may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence-based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity, and as such sequences would generally be considered “identical.”

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

### Sample Processing Methods

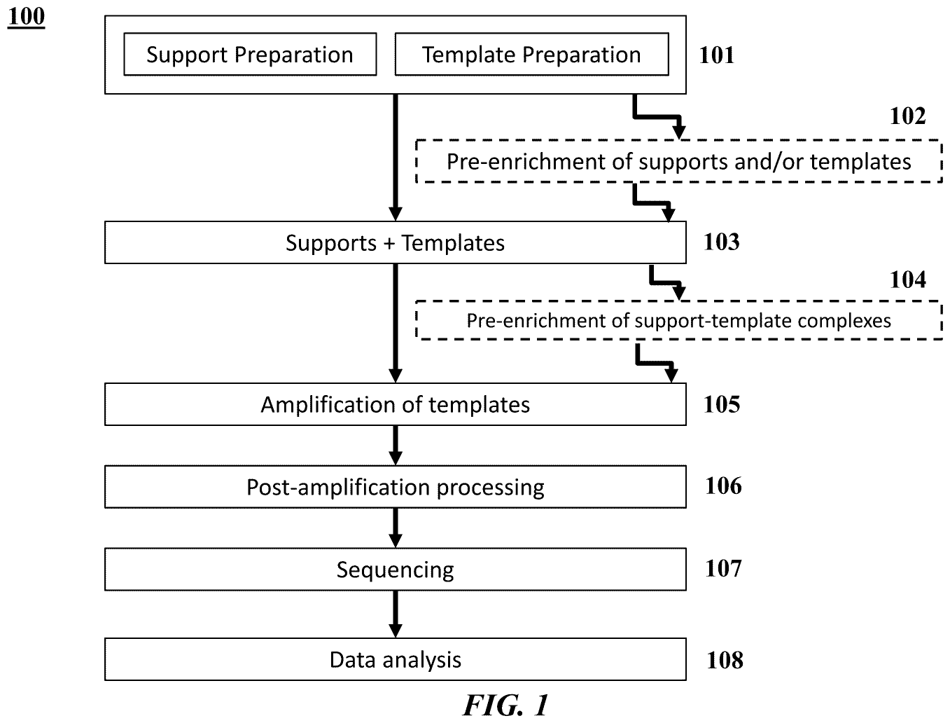

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., a bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture, or be coupled to, a template nucleic acid (or a processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., a splint, a primer, a template nucleic acid, a capture sequence, and the like), or any other functional sequence useful for a downstream operation, or any combination thereof. The oligonucleotide may be single-stranded, double-stranded, or partially double-stranded.

A support may comprise one or more capture entities, where a capture entity is configured for capture by a capturing entity. A capture entity may be coupled to an oligonucleotide coupled to the support. A capture entity may be coupled to the support. For example, the capturing entity may comprise streptavidin (SA) when the capture entity comprises biotin. In another example, the capturing entity may comprise a complementary capture sequence when the capture entity comprises a capture sequence (e.g., a capture oligonucleotide that is complementary to the complementary capture sequence). In another example, the capturing entity may comprise an apparatus, system, or device configured to apply a magnetic field when the capture entity comprises a magnetic particle. In another example, the capturing entity may comprise an apparatus, system, or device configured to apply an electrical field when the capture entity comprises a charged particle. In some instances, the capturing entity may comprise one or more other mechanisms configured to capture the capture entity. A capture entity and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively, or in addition, a single capturing entity may be capable of associating with multiple capture entities. The capture entity may be capable of linking to a nucleotide. Chemically modified bases comprising biotin, an azide, cyclooctyne, tetrazole, and a thiol, and many others are suitable as capture entities. The capture entity/capturing entity pair may be any combination. The pair may include, but is not limited to, biotin/streptavidin, azide/cyclooctyne, and thiol/maleimide. It will be appreciated that either of the pair may be used as either the capture entity or the capturing entity. In some instances, the capturing entity may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in U.S. Patent Application Publication No. US20220042072A1 and PCT Publication No. WO2022040557A2, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprise capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprise cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR), or in wells. Emulsion PCR methods are described in further detail U.S. Patent Application Publication No. US20220042072A1 and PCT Publication No. WO2022040557A2, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. Nos. 10,900,078 and 11,118,223, and PCT Publication No. WO2022040557A2, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate, such as via a support or otherwise. Examples of substrate-based sample processing systems are described elsewhere herein. Any sequencing method described elsewhere herein may be used, for example pyrosequencing, single molecule sequencing, sequencing by synthesis (SBS), sequencing by ligation, sequencing by binding, etc. In some cases, sequencing by synthesis (SBS) is performed. In some cases, sequencing by a flow-based sequencing method is performed.

For example, sequencing comprises extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated by reference herein for all purposes, and described further below. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g. sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g. [A T G C]), where each nucleotide is not terminated, and where each base is labeled with a same dye (yielding same frequency optical signals). With each flow, other sequencing reagents, e.g. sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot. Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, 30rythrosine, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q, 532, and 633, or other fluorophores and quenchers. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. In some cases, the sequencing reads may be processed to generate spatial data.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Patent Pub. No. 20200326327A1, U.S. Patent Pub. No. 20210079464A1, International Patent Pub. No. WO2022072652A1, U.S. Patent Pub. No. 20210354126A1, and International Patent Pub. No. WO2023192403A2, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (pm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

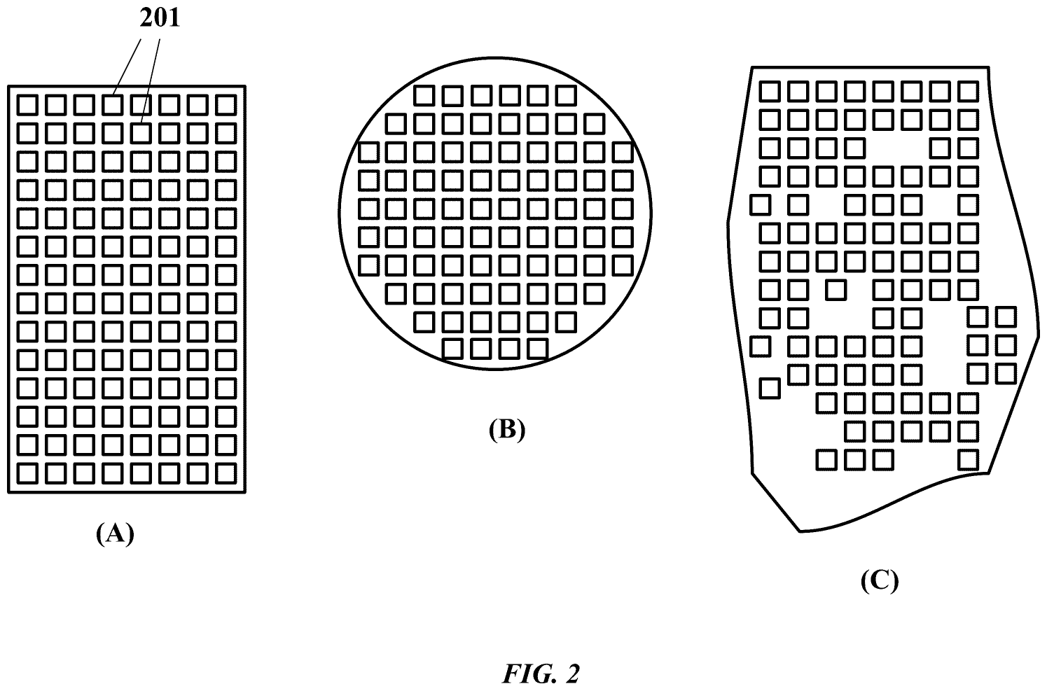

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

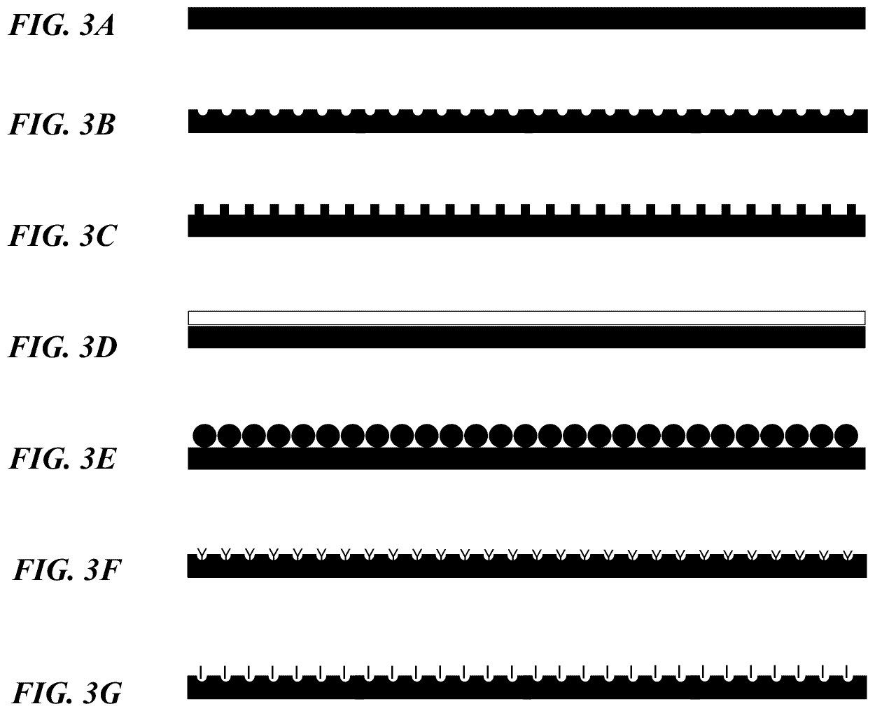

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

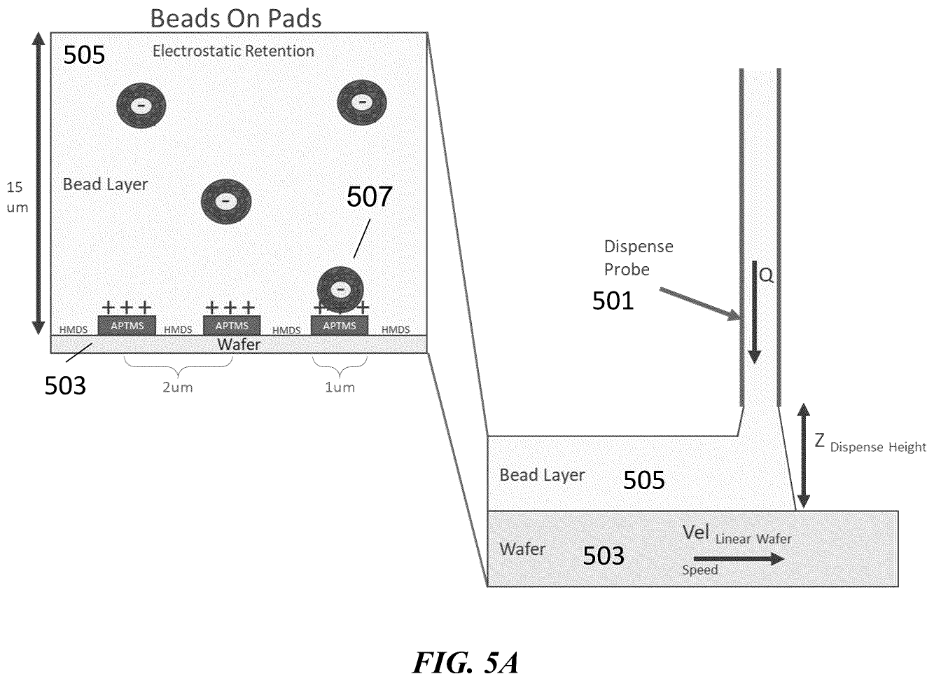

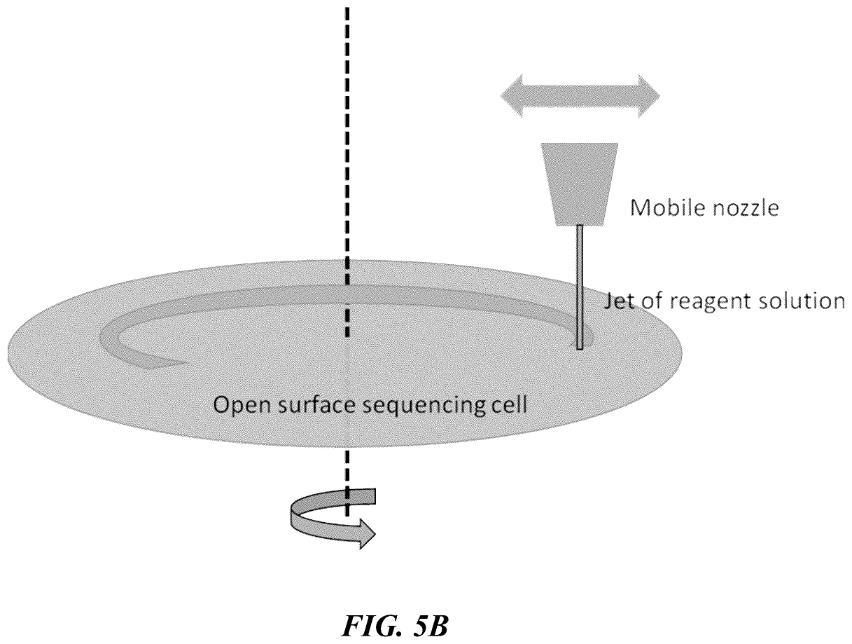

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at a rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

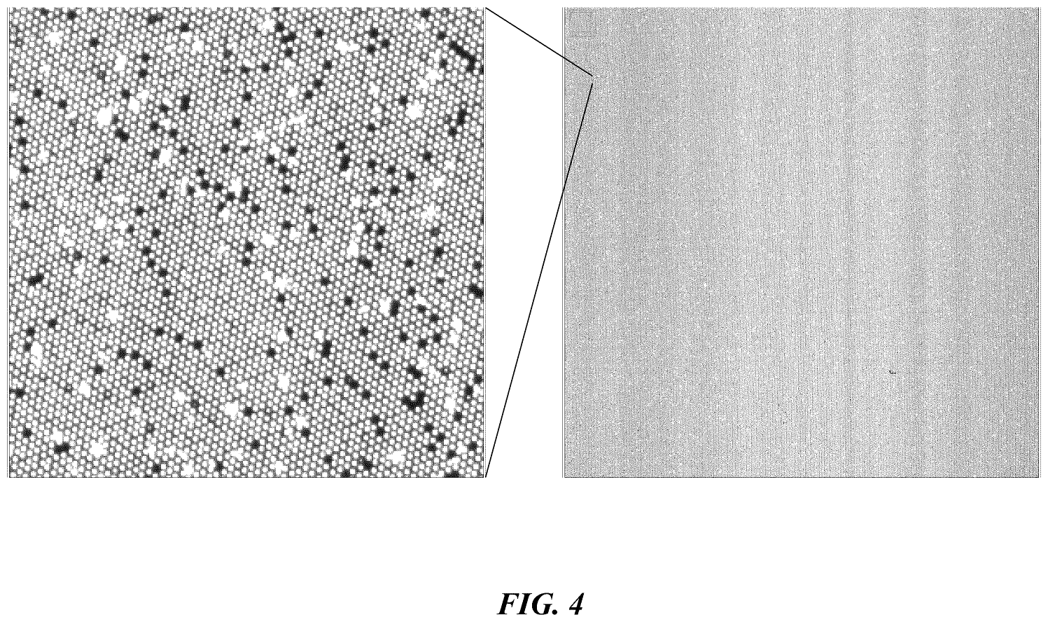

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high-speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g. interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g. the enclosure can retain the fluid via surface tension). In some cases, an electric field may be used to regulate a hydrophobicity of one or more surfaces of the container to retain at least a portion of the fluid contacting the immersion objective lens and the open substrate

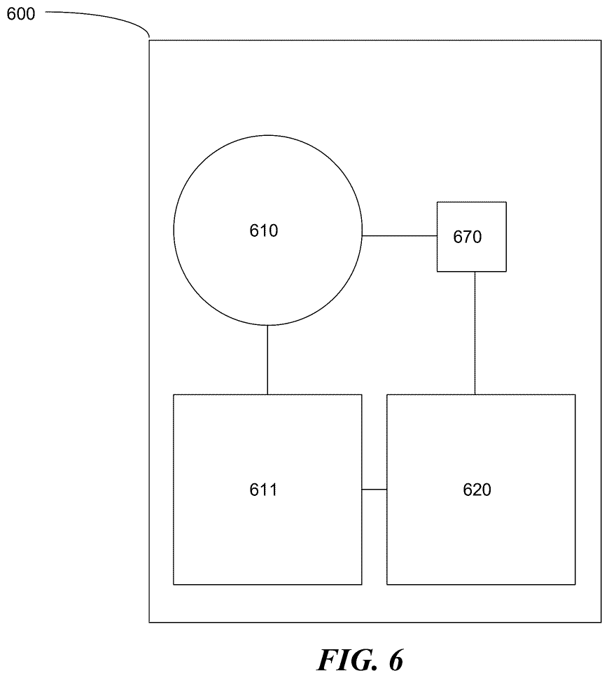

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in Patent Pub. No. US20210354126A1, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

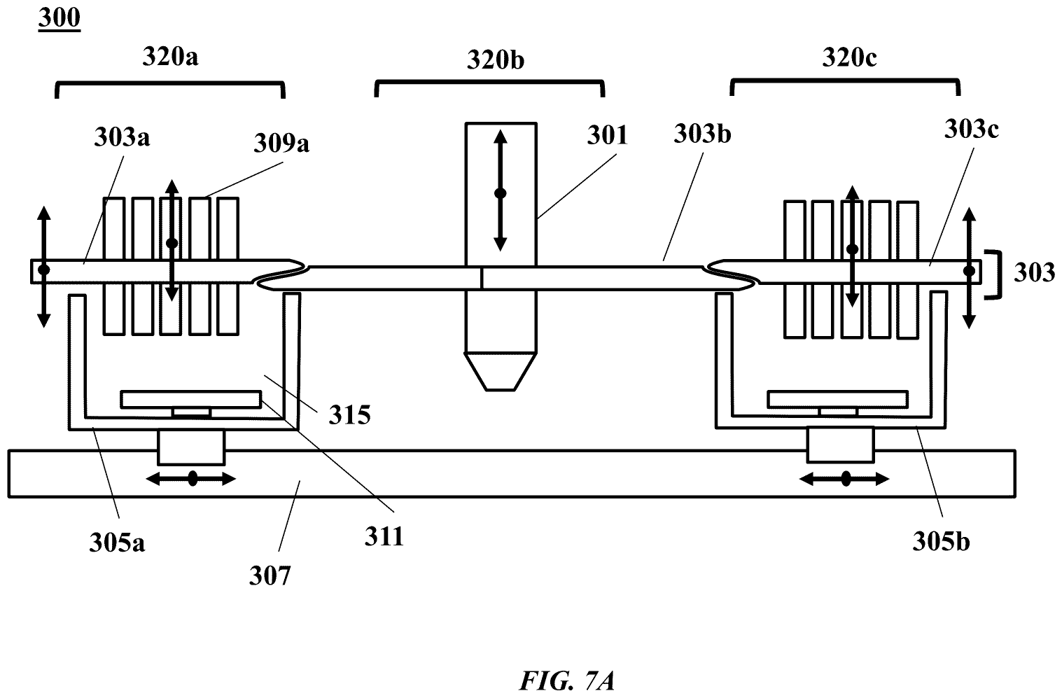

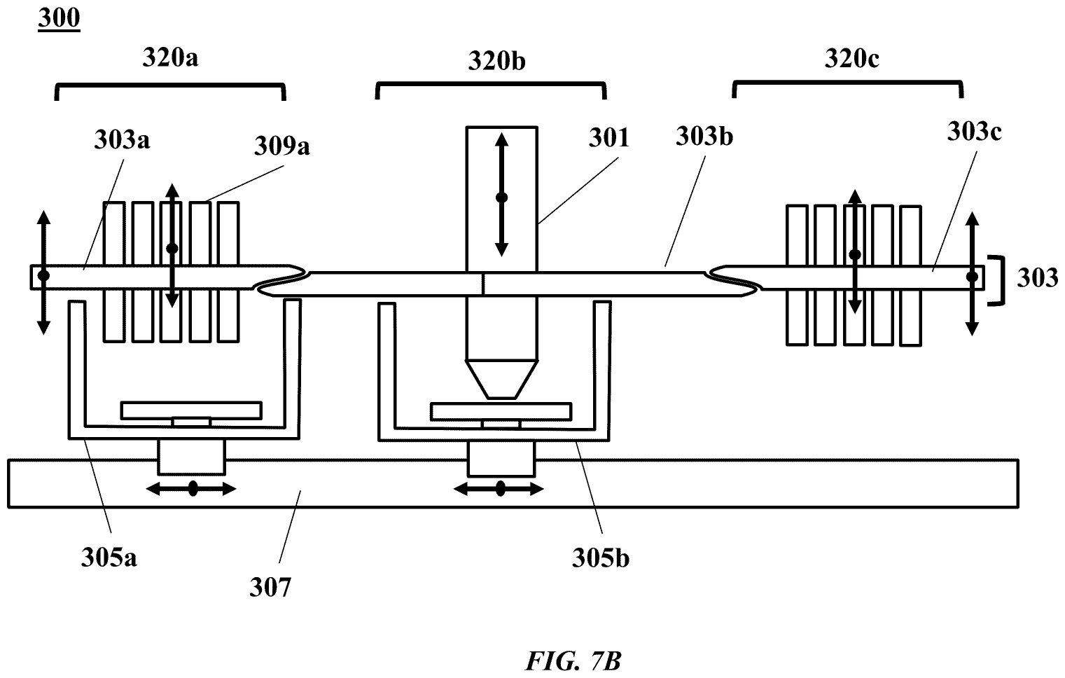

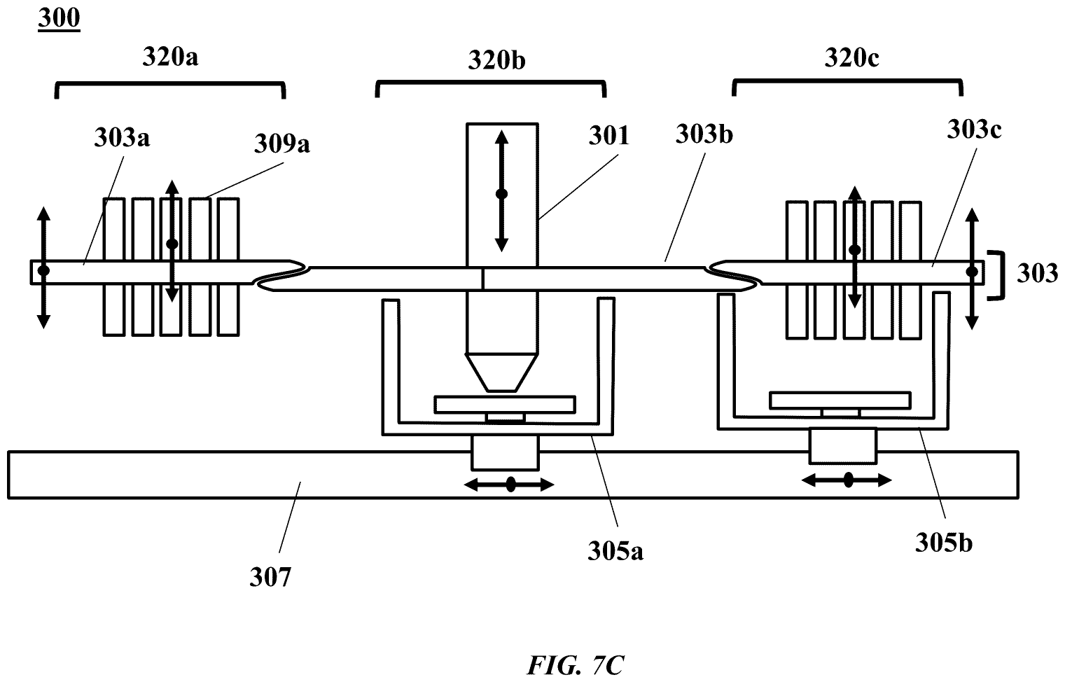

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311, as shown in FIG. 7A) in a first sample environment system (e.g. 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Probes for Flow-Based Sequencing

Provided herein are devices, systems, methods, compositions, and kits for multiplexed measurements using oligonucleotide probes encoded for flow-based sequencing. An oligonucleotide probe may be configured to attach, directly or indirectly, to a target analyte, such as a transcript or protein. For example, the oligonucleotide probe may be conjugated to an antibody, which antibody binds to a target protein. In another example, the oligonucleotide probe may bind to a target transcript by complementary binding. The oligonucleotide probe may be sequenced while attached to the target analyte or detached from the target analyte. In some cases, a derivative, such as an extension product, a reverse complement, and/or amplicon of the oligonucleotide probe may be sequenced. An oligonucleotide probe may comprise a flow-based code domain comprising a nucleic acid sequence encoding a flow-space sequence, a primer binding site, and a target-related domain. In some cases, the flow-space sequence may be configured to generate a flowgram unique to the probe amongst a plurality of probes during flow-based sequencing, wherein the flowgram comprises a set of relative intensity values generated during the flow-based sequencing. Such devices, systems, methods, compositions, and kits may be applied in conjunction with, alternatively, or in addition to one or more operations described in the sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

### Sequencing Methods

During sequencing by synthesis, a sequencing primer may be hybridized to a template (e.g., to a primer binding site on the template) and extended in a stepwise manner by, in each extension step, contacting the complex with nucleotide reagents of known canonical base type(s). The extended or extending sequencing primer may also be referred to herein as a growing strand. An extension step may be a bright step (also referred to herein, in some cases, as labeled step, hot step, or detected step) or a dark step (also referred to herein, in some cases, as an unlabeled step, cold step, or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted. Sequencing data can be generated from the signals collected after one or more extension steps. A sequencing by synthesis method may comprise any number of bright steps and any number of dark steps. A sequencing by synthesis method may comprise any number of bright regions (consecutive bright steps) and any number of dark regions (consecutive dark steps). In some cases, the dark steps or dark regions may be used to accelerate or fast forward through certain regions of the template during sequencing. In some cases, the dark steps or dark regions may be advantageous to correct phasing problems.

Sequencing methods of the present disclosure may comprise flow-based sequencing, non-terminated sequencing, and/or terminated sequencing. Sequencing methods of the present disclosure may be applied to colony-based sequencing where template strands are provided in clusters, each cluster comprising copies of a single template strand, concatemer-based sequencing where template strands are provided as concatemers, each concatemer comprising multiple copies of a single template insert, or single molecule-based sequencing where template strands are provided as single molecules as opposed to colonies, clusters, or concatemers. For non-single molecule-based sequencing methods, multiple sequencing primers may be simultaneously bound to multiple primer binding sites across multiple copies of a template insert (in clusters or in a concatemer), extended in parallel, and provide synchronized and cumulative signals from the multiple copies at bright steps.

### Terminated Sequencing

In terminated sequencing methods, a bright step may comprise terminated nucleotides (e.g., reversibly terminated nucleotides). In some cases, a bright step may comprise a single nucleotide base type (e.g., A, C, G, T, U) or a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) at most a single nucleotide base may be incorporated into a growing strand. In an extension step comprising a mixture of reversibly terminated and unterminated nucleotides, more than one nucleotide base may be incorporated into a growing strand, the last incorporation being of a terminated nucleotide.

### Non-Terminated Sequencing, Flow-Based Sequencing

Sequencing data can be generated using a flow-based sequencing method that includes extending a primer bound to a template polynucleotide molecule according to a pre-determined flow cycle where, in any given flow position, a single type of nucleotide is accessible to the extending primer. In some embodiments, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template polynucleotide molecule. In some embodiments, for example, sequencing data is generated using a flow-based sequencing method that includes extending a primer using labeled nucleotides, and detecting the presence or absence of a labeled nucleotide incorporated into the extending primer. Flow-based sequencing methods may also be referred to as “natural sequencing-by-synthesis,” “mostly natural sequencing-by-synthesis,” or “non-terminated sequencing-by-synthesis” methods. Exemplary methods are described in U.S. Pat. No. 8,772,473, which is incorporated herein by reference in its entirety. While the following description is provided in reference to flow-based sequencing methods, it is understood that other sequencing methods may be used to sequence all or a portion of a sequenced region.

Flow-based sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (e.g., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

Flow-based sequencing may be used to sequence an oligonucleotide probe, or its derivative, comprising a “flow-based code domain”. The flow-based code domain may comprise a nucleic acid sequence encoding a “flow-space sequence”. The flow-space sequence of a particular nucleic acid sequence may be the sequence of integers that represent the relative signals measured for a sequence of flows interrogating the particular nucleic acid sequence in a specific flow order. The flow-space sequence may be represented as a flow chart or flowgram (e.g., [1 1 0 1] for four flows). Using the flow-space sequence, and the specific flow order as a key, one can determine the nucleic acid base sequence.

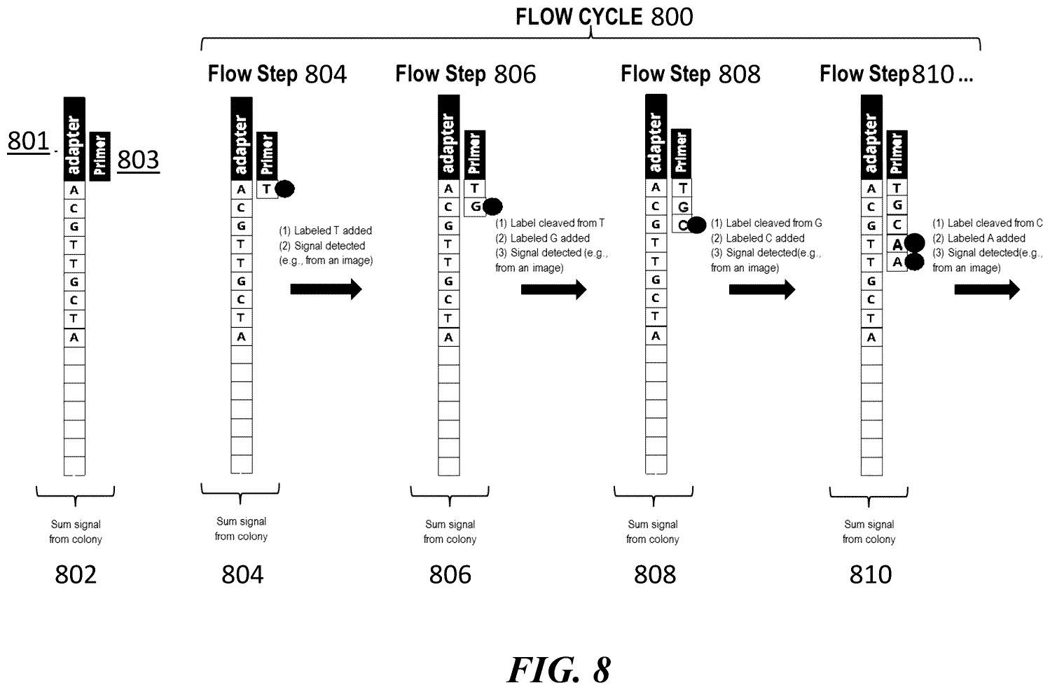

FIG. 8 illustrates an exemplary flow-based sequencing method that can be used to generate the sequencing data described herein. In some embodiments, polynucleotides may be bound to a surface (e.g., the surface of a bead attached to a substrate), as described in detail herein. The polynucleotides can include a nucleic acid sequence of interest (also referred to as a “template sequence”) and can further include a sequencing adapter sequence. The nucleic acid sequence of interest can be a nucleic acid molecule from or derived from a sample of a subject.

In the depicted example in FIG. 8, the nucleic acid sequence of interest includes an adaptor sequence 801 followed by the nucleic acid sequence of interest (“ACGTTGCTA . . . ”). The adapter sequence 801 can include a sequencing primer hybridization site. At step 802, a sequencing primer 803 is hybridized to the adapter sequence 801 of the polynucleotide at the sequencing primer hybridization site.

The sequencing primer is then extended in a series of flow cycles. In a flow cycle, the hybrid (e.g., the polynucleotide adapter hybridized to the sequencing primer) is combined with nucleotides (e.g., at least partially labeled nucleotides) and one or more signals indicating nucleotide incorporation into the sequencing primer may be detected. In the depicted example, the flow cycle 800 includes four flow steps 804, 806, 808, and 810. In a given flow step, a single type of nucleobase is combined with the hybrid according to the flow-cycle order T-G-C-A. As shown in FIG. 8, in flow step 804, labeled T nucleotides are combined with the hybrid; in flow step 806, labeled G nucleotides are combined with the hybrid; in flow step 808, labeled C nucleotides are combined with the hybrid; in flow step 810, labeled A nucleotides are combined with the hybrid.

At 804, labeled T nucleotides are combined with the hybrid. Since the T base is complementary to the A base in the template polynucleotide, it is incorporated into the extending primer to form the hybrid as shown in 804. Further, a signal indicative of the incorporation of labeled T nucleotide into the sequencing primer can be detected. The signal may be detected, for example, by imaging the surface the polynucleotides are deposited on and analyzing the resulting image(s). In some embodiments, the sequencing platform may be washed with a wash buffer to remove unincorporated nucleotides prior to signal detection. In some embodiments, the detection of the signal is based on image processing techniques described herein.

At step 806, the label may be removed from the T nucleotide (e.g., by cleaving the label from the nucleotide). The sequencing method can then be continued with the next base in the flow order, G in the example illustrated in FIG. 8. At step 806, labeled G nucleotides are combined with the hybrid. Since the G base is complementary to the C base in the template polynucleotide, it is incorporated to form the hybrid in 806. Further, a signal indicating the incorporation of the labeled G nucleotide can be detected.

At step 808, the label may be removed from the G nucleotide (e.g., by cleaving the label from the nucleotide). The sequencing method can then be continued with the next base in the flow order, C. At step 808, labeled C nucleotides are combined with the hybrid. Since the C base is complementary to the G base in the template polynucleotide, it is incorporated into the extending primer to form the hybrid in 808. Further, a signal indicating the incorporation of the labeled C nucleotide into the sequencing primer can be detected.

At step 810, the label may be removed from the C nucleotide (e.g., by cleaving the label from the nucleotide). The sequencing method can then be continued with the next base in the flow order, A. At step 810, labeled A nucleotides are combined with the hybrid. Since the A base is complementary to the T base in the template polynucleotide, it is incorporated into the extending primer to form the hybrid in 810. Further, a signal indicating the incorporation of the labeled A nucleotide into the sequencing primer can be detected. In step 810, because the template sequence includes two consecutive T bases, two A nucleotides are incorporated into the extending sequencing primer. Thus, the detected signal intensity indicating the incorporation of two A nucleotides may be greater than the signal intensity indicating the incorporation of one nucleotide.

For simplicity, FIG. 8 illustrates incorporation of two labeled A nucleotides in the same hybrid. However, flow-based sequencing may be performed on colonies of amplified molecules, e.g., each bead representing one colony, where an optically resolvable location contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

While each flow step in the exemplary flow-based sequencing method in FIG. 8 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template polynucleotide). For example, if C nucleotides are combined with a hybrid having a C base, no incorporation would occur and thus no signal indicative of an incorporation would be detected. Further, as shown in step 810, two nucleotides or more than two nucleotides may be incorporated into the sequencing primer for larger homopolymer lengths in the nucleic acid sequence of interest.

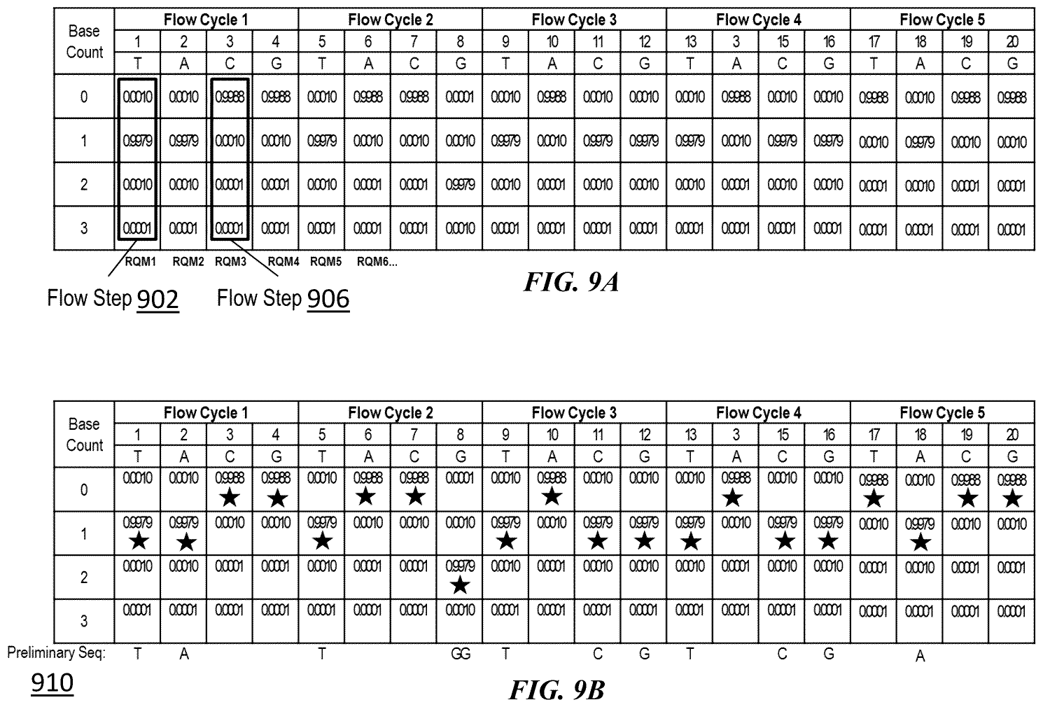

FIGS. 9A-9B illustrate exemplary detected signals and corresponding determined sequence after five exemplary flow cycles are performed. FIG. 9A shows an exemplary summary of detected signals after five exemplary flow cycles are performed, in accordance with some embodiments. Solely by way of example, a primer extended using a repeating flow-cycle order of T-A-C-G may result in a sequencing data “flowgram” set shown in FIG. 9A. Each column in FIG. 9A corresponds to a flow step and the values in each column collectively represent the detected signal intensity in the corresponding flow step, as described below. Thus, the flowgram may include a set of relative intensity values generated during the flow-based sequencing. A flow-based code domain of a probe may be configured to generate a flowgram unique to the probe amongst a plurality of probes during flow-based sequencing. In some cases, the flowgram comprises a set of relative intensity values generated during the flow-based sequencing.

In each flow step, the flow signal can be determined from an analog signal that is detected during the sequencing process, such as a fluorescent signal of the one or more bases incorporated into the sequencing primer during sequencing. Although an integer number of zero or more bases are incorporated at any given flow position, a given analog signal many not perfectly match with the analog signal. Therefore, in some embodiments, for a given flow step (e.g., flow step 902), the detected signal intensity can be expressed in probabilistic terms. Specifically, the detected signal intensity can be expressed in four likelihood values corresponding to 0 base, 1 base, 2 bases, and 3 bases, respectively.

In the depicted example, for flow step 902, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In the depicted example, the incorporation is a T since the flow step introduced labeled T nucleotides, which means there is an A in the template.

On the other hand, in flow step 906, the detected signal intensity is expressed by a first likelihood value of 0.9988 for 0 base, a second likelihood value of 0.001 for 1 base, a third likelihood value of 0.001 for 3 bases, and a fourth likelihood value of 0.0001 for 4 bases. This can be interpreted to indicate that there is a high likelihood that no nucleotide base has been incorporated. In the depicted example, no C has been incorporated.

Accordingly, the flowgram set in FIG. 9A is formatted as a sparse matrix, with a flow signal represented by a plurality of likelihood values indicating a plurality of likelihoods for a plurality of base homopolymer length counts (e.g., 0 base count, 1 base count, 2 base counts, and 3 base counts) at each flow position.

The homopolymer length likelihood may vary, for example, based on the noise or other artifacts present during detection of the analog signal during sequencing. In some embodiments, if the homopolymer length likelihood statistical parameter or likelihood is below a predetermined threshold, the parameter may be set to a predetermined non-zero value that is substantially zero (e.g., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0).

With reference to FIG. 9B, a preliminary sequence can be determined based on the flowgram in FIG. 9A. For example, the most likely sequence can be determined by selecting the base count with the highest likelihood at each flow position, as shown by the stars in FIG. 9B. The flowgram as depicted in FIGS. 9A-9B may be re-formatted (from a sparse matrix format, with a plurality of likelihood values at each flow position) to a one-dimensional matrix format, with integer values at each flow position, the integer representing the base count with the highest likelihood value at each flow position: e.g., [1 1 0 0 1 0 0 2 1 0 1 1 1 0 1 1 0 1 0 0]. Thus, the preliminary sequence 910 can be determined as: TATGGTCGTCGA (SEQ ID NO:2). From the preliminary sequence (e.g., preliminary sequence 910), the reverse complement (e.g., the template strand or the nucleic acid sequence of interest) can be readily determined. Further, the likelihood of this sequencing data set, given the TATGGTCGTCGA (SEQ ID NO:2) sequence (or the reverse complement), can be determined as the product of the selected likelihood at each flow position.

The signal for any flow position in the sequencing data is flow-order-dependent in that the flow order used to sequence the polynucleotide at any base position can affect the flow signal at that position. Random fragmentation of nucleic acid molecules (either in vivo fragmentation, such as cell-free DNA, or in vitro fragmentation, such as by sonication or enzymatic digestion) that overlap at the same locus results in multiple different sequencing start sites (relative to the locus) for the nucleic acid molecules.

The nucleotides can be introduced at a determined order during the course of primer extension, which may be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. Further, one or more cycles may omit one or more nucleotides. Solely by way of example, the order of a first cycle may be A-T-G-C and the order of a second cycle may be A-T-C. Alternative orders may be readily contemplated by one skilled in the art. Between the introductions of different nucleotides, unincorporated nucleotides may be removed, for example by washing the sequencing platform with a wash fluid.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Sequencing data, such as a flowgram as described below, can be generated based on the detection of an incorporated nucleotide and the order of nucleotide introduction. For example, a flowgram for the following template sequences is shown in Table 1: CTG and CAG, and a repeating flow cycle of T-A-C-G (that is, sequential addition of T, A, C, and G nucleotides, which would be incorporated into the primer only if a complementary base is present in the template polynucleotide). In Table 1, 1 indicates incorporation of an introduced nucleotide, 0 indicates no incorporation of an introduced nucleotide, and an integer x>1 indicates incorporation of x introduced nucleotides. The flowgram can be used to determine the sequence of the template strand (e.g., the sequence of the template strand may be considered as the complement of the incorporated nucleotides). The flowgram of Table 1 is formatted as a one-dimensional matrix format, with an integer at each flow position (flow step).

TABLE 1

| Flow Cycle | |
|---|---|
| 1 | 2 |

Cycle Step

| | | | | | | | |
|---|---|---|---|---|---|---|---|
| 1 | 2 | 3 | 4 | 1 | 2 | 3 | 4 |

Flow Bases

| | | | | | | | |
|---|---|---|---|---|---|---|---|
| T | A | C | G | T | A | C | G |

Sequence | Number of Bases Incorporated

| | | | | | | | | |
|---|---|---|---|---|---|---|---|---|
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

A flowgram may be binary or non-binary. A binary flowgram detects the presence (1) or absence (0) of an incorporated nucleotide. A non-binary flowgram, such as shown in Table 1, can more quantitatively determine a number of incorporated nucleotides from each stepwise introduction. A non-binary flowgram also indicates the presence or absence of the base, but can provide additional information including the number of bases incorporated at the given step. For example, the sequence of CCG would incorporate two G bases in one flow cycle step (e.g., in flow cycle 1, cycle step 4), and any signal emitted by the two labeled bases would have a greater intensity than the incorporation of a single base.

Prior to generating the sequencing data, the polynucleotide is hybridized at a hybridization site to a sequencing primer to generate a hybridized template. The polynucleotide may be ligated to an adapter during sequencing library preparation, such as during the attachment of one or more barcode regions to the polynucleotide. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Pat. No. 10,344,328 and International patent application WO 2020/227143, each of which is incorporated herein by reference in its entirety.

### Re-sequencing

In some cases, a method for sequencing may comprise sequencing a same template strand multiple times to generate robust sequencing data (e.g., a high quality sequencing read) corresponding to the template strand. In some cases, a method for sequencing may comprise sequencing a same template strand multiple times and sequencing a same reverse complement strand of the template strand multiple times (e.g., both forward and reverse strands) to generate robust sequencing data (e.g., a high quality paired end read) corresponding to the template strand. A method for re-sequencing a template strand (which may be a forward strand or reverse strand) may comprise annealing a first sequencing primer to the template strand, extending the first sequencing primer through at least a first portion of the template strand via any combination of bright steps and/or dark steps to generate first sequencing data, denaturing the extended strand from the template strand, annealing a second sequencing primer to the template strand, and extending the second sequencing primer through at least a second portion of the template strand via any combination of bright steps and/or dark steps to generate second sequencing data, and processing (e.g., combining, comparing, matching, aligning, resolving, etc.) the first sequencing data and the second sequencing data to generate a sequencing read of the template strand. A template strand may be denatured and re-sequenced any number of times, such as about, at least about, and/or at most about 1, 2, 3, 4, 5, 6, 7, 8, 9, 10 or more times, such as by annealing an nth sequencing primer to the template strand and extending the nth sequencing primer through at least an nth portion of the template strand. The different n sequencing primers may comprise the same or different sequences which may bind to same or different primer binding sites on the template strand, respectively. The different nth portions on the template strand may refer to the same portions or different portions on the template strand. Two portions on the template strand (that are extended through) may be partially overlapping, completely overlapping (for one or both portions), or non-overlapping. The respective extensions through the template strand in the different sequencing runs may use the same or different nucleotide reagents (e.g., non-terminated nucleotides during a first sequencing run, terminated during a second sequencing run; green dye-labeled nucleotides during a first sequencing run, red dye-labeled nucleotides during a second sequencing run; labeled A-, T-, G-bases and unlabeled C-base nucleotides during a first sequencing run, labeled A-, T-, C-bases and unlabeled G-base nucleotides during a second sequencing run; 5% labeled A bases during a first sequencing run; 100% labeled A bases during a second sequencing run; etc.). The respective extensions through the template strand in the different sequencing runs may have the same flow order or flow cycle of nucleotide reagents. The respective extensions through the template strand in the different sequencing runs may have different flow orders or flow cycles of nucleotide reagents (e.g., A->T->G->C single base flow cycle order during a first sequencing run, T->A->G->C single base flow cycle order during a second sequencing run; A/T/G/C 4-base flow cycle order during a first sequencing run; A/T/G->A/T/C 3-base flow cycle order during a second sequencing run, etc.). Denaturing may comprise contacting the double-stranded nucleic acid molecule with denaturing agents, such as sodium hydroxide (NaOH) or ethylene carbonate. An entire substrate may be subjected to resequencing by, after a first sequencing run, contacting the entire surface with a solution comprising a denaturing agent, contacting the entire surface with a solution comprising sequencing primers under conditions sufficient to anneal them to template nucleic acid strands immobilized to the substrate, and subjecting them to extension reactions. In some cases, denaturing may comprise applying heat to the double-stranded nucleic acid molecule.

### Spatial Screening

The systems, devices, and methods described herein may be useful for various spatial screening applications, such as to determine or identify information related to a spatial resolution within or of an analyte and/or sample.

Disclosed are systems and methods that may be used to carry out omic-based studies, such as the characterization, measurement, and/or quantification of analytes of biological samples, with location information, using systems and methods described herein. In some cases, omic-based studies, such as the characterization, measurement, and/or quantification of analytes, may comprise genomics, epigenomics, lipidomics, proteomics, glycomics, transcriptomics, metabolomic, microbiomics, connectomics, or any derivatives thereof. In some cases, omic-based approaches may study, characterize, measure, and/or quantify analytes or derivatives thereof of biological samples. An analyte may be or comprise any analyte described herein. In some instances, an analyte may comprise a nucleic acid, polypeptide, protein, carbohydrate, lipid, derivatives thereof, or any combinations thereof. A nucleic acid may be or comprise any nucleic acid molecule described herein. In some cases, a nucleic acid may comprise deoxyribonucleic acid (DNA), ribonucleic acid (RNA), locked nucleic acid (LNA), peptide nucleic acid (PNA), modified nucleic acid (XNA), derivative thereof, or any combination thereof. In some cases, a nucleic acid may comprise an endogenous or exogenous chemical modification. Such a modification may comprise a sugar modification, a base modification, a backbone modification, or an unnatural base pairing. For example, DNA may be methylated or acetylated. In some cases, a carbohydrate may comprise a monosaccharide, a polysaccharide, a lignin, derivatives thereof, or any combinations thereof. In some cases, a peptide or a protein may be modified. A modification of a protein or a peptide, in some cases, may comprise an ubiquitylation, sumolyation, ubiquitin-like modification, phosphorylation, methylation, acetylation, proteolysis, glycosylation, isoprenylation, deamidation, eliminylation, AMPlyation, ADP-ribosylation, redox, any derivatives thereof, or any combinations thereof.

In some cases, the analyte may include an antibody or binding fragment thereof, an oligonucleotide, an RNA transcript, a protein, a polypeptide, a metabolite, or genomic DNA.

A biological sample for spatial screening may be or comprise any biological sample described herein. In some cases, a biological sample may comprise a single cell, a group of cells, a tissue, an organ, or derivatives thereof. For example, a cell may comprise a blood cell or a tumor cell. In some cases, a biological sample may comprise a tissue or a portion thereof obtained by a biopsy. In some cases, a biological sample may be in a healthy state. In other cases, a biological sample may be in a disease state when compared to another biological in a healthy state. In some cases, a biological sample may comprise a tumor. In some instances, a biological sample may comprise multiple cells from the same organism. In other cases, a biological sample may comprise multiple cells from more than one organism. In one case, a biological sample may comprise an infected tissue. In instances, a biological sample may originate in in vitro culture. One such example may comprise an organoid.

In some cases, a biological sample may comprise a plurality of cells or tissue samples. A plurality of cells or tissue samples, in some cases, may comprise at least about 1×10² cells or tissue samples, at least about 1×10³ cells or tissue samples, at least about 1×10⁴ cells or tissue samples, at least about 1×10⁵ cells or tissue samples, at least about 1×10⁶ cells or tissue samples, at least about 1×10⁷ cells or tissue samples, at least about 1×10⁸ cells or tissue samples, at least about 1×10⁹ cells or tissue samples, at least about 1×10¹⁰ cells or tissue samples, at least about 1×10¹¹ cells or tissue samples, or at least about 1×10¹² cells or tissue samples. In other cases, a plurality of cells or tissue samples may comprise from about 5×10¹ to about 1×10² cells or tissue samples, from about 1×10² to about 5×10² cells or tissue samples, from about 5×10² to about 1×10³ cells or tissue samples, from about 1×10³ to about 5×10³ cells or tissue samples, from about 5×10³ to about 1×10⁴ cells or tissue samples, from about 1×10⁴ to about 5×10⁴ cells or tissue samples, from about 5×10⁴ to about 1×10⁵ cells or tissue samples, from about 1×10⁵ to about 5×10⁵ cells or tissue samples, from about 5×10⁵ to about 1×10⁶ cells or tissue samples, from about 1×10⁶ to about 5×10⁶ cells or tissue samples, from about 5×10⁶ to about 1×10⁷ cells or tissue samples, from about 1×10⁷ to about 5×10⁷ cells or tissue samples, from about 5×10⁷ to about 1×10⁸ cells or tissue samples, from about 1×10⁸ to about 5×10⁸ cells or tissue samples, from about 5×10⁸ to about 1×10⁹ cells or tissue samples, from about 1×10⁹ to about 5×10⁹ cells or tissue samples, from about 5×10⁹ to about 1×10¹⁰ cells or tissue samples, from about 1×10¹⁰ to about 5×10¹⁰ cells or tissue samples, from about 5×10¹⁰ to about 1×10¹¹ cells or tissue samples, from about 1×10¹¹ to about 5×10¹¹ cells or tissue samples, or from about 5×10¹¹ to about 1×10¹² cells or tissue samples.

In some instances, a cell or other sample may be deconstructed into individual analytes after each analyte is encoded with a location. Such deconstruction may allow the analytes to be processed by the systems and methods described herein. Such deconstruction may also facilitate or improve the processing by the systems and methods described herein. Once processed, the analytes can be reconstructed (e.g., spatially reconstructed) by detecting and digitally decoding the location using molecular probes encoding the respective locations of the analytes. In other cases, a biological sample may not undergo deconstruction to be processed by the systems and methods described herein.

The location of an analyte may comprise the spatial origin information of the analyte in a biological sample. The location of an analyte may comprise any location information of the analyte with respect to a biological sample. In some cases, a location may be a two-dimensional location. In other cases, a location may be a three-dimensional location. A location of an analyte may be encoded, such as via molecular encoding or digital encoding.

Encoding a location may comprise recording or specifying an addressable location of an analyte with a spatial reference system. A spatial reference system may comprise a Cartesian coordinate system (e.g., comprising 2 axes or 3 axes). A 2-axis coordinate system may specify an analyte on a two-dimensional space. A 3-axis coordinate system may specify an analyte on a three-dimensional space. In some cases, a spatial reference system may also comprise a pixel of an image. A spatial reference system may comprise any other coordinate system (e.g., polar coordinate system). A spatial reference system may comprise a reference point (e.g., origin, or non-origin point).

Molecular encoding of a location of an analyte may comprise linking a molecular probe encoding a location to the analyte. Linking a molecular probe to an analyte may comprise, for example, formation of a covalent or non-covalent bond, a binding between the molecular probe and the analyte, a hybridization between the molecular probe and the analyte, and/or generating a derivative of the analyte using the molecular probe. A molecular probe may comprise a barcode sequence. A molecular probe may comprise a flow-based code domain.

In some cases, a molecular probe encoding a location of an analyte may be the same molecular species as the analyte (e.g., a nucleic acid molecule encoding the location information of another nucleic acid molecule). In some cases, a molecular probe encoding a location of an analyte may be the derivative of the analyte (e.g., a DNA molecule encoding the location information of a cDNA molecule reverse transcribed from an RNA molecule). In other cases, a molecular probe encoding a location of an analyte may be a different molecular species from the analyte (e.g., a nucleic acid molecule encoding the location information of a peptide molecule). In some instances, a molecular probe encoding a location of an analyte may comprise one molecule. In some cases, a molecular probe encoding a location of an analyte may comprise more than one molecule. In some cases, a molecular probe encoding a location of an analyte may comprise 2, 3, 4, 5, 6, 7, 8, 9, 10 or more molecules.

In some cases, a molecular probe encoding a location of an analyte may encode the location of only the analyte. In other cases, a molecular probe encoding a location of an analyte may also encode the location of another analyte. For example, the location of an analyte may be used to decode the location of its neighboring analyte or otherwise an additional analyte within proximity. In some cases, a molecular probe encoding a location of an analyte may also encode the spatial information of analytes sharing the same molecular origin. For example, a molecular probe of a nucleic acid of a cell can encode the location information of other nucleic acids from the same cell. In some instances, an analyte and a molecular probe encoding a location information of the analyte may originate from the same sample. In some cases, an analyte and a molecular probe encoding a location of the analyte may originate from two different samples. For example, the expression level of a marker nucleic acid in one biological sample may be used to identify a single cell based on the expression of the marker gene from the single cell sequencing result of another biological sample. In some cases, when an analyte and a molecular probe encoding a location of the analyte originate from two different samples, the analysis of the location of the analyte may comprise use of an algorithm.

A molecular probe encoding a location of an analyte may comprise a flow-based code domain, a barcode sequence, a peptide sequence, a hybridization pattern, a fluorescence measurement, a fluorophore identification, an enzymatic reaction, derivatives thereof, or any combination thereof.

In some cases, encoding a location of an analyte may occur before the analyte is amplified. In some cases, encoding a location of an analyte may occur after the analyte is amplified. In some cases, encoding a location of an analyte may occur simultaneously when the analyte is amplified. In some cases, encoding a location of an analyte may occur without the analyte being amplified. In some cases, a molecular probe encoding a location of an analyte may be amplified. In other cases, a molecular probe encoding a location of an analyte may not be amplified. In some cases, an amplification reaction or process may comprise any amplification reaction or process described herein. In some instances, an analyte or a molecular probe encoding a location of the analyte may also be extended, ligated, cleaved, circularized, linearized, or activated. In some instances, a photoactivation may be required to extend, ligate, cleave, or activate an analyte or a molecular probe encoding a location of the analyte.

In some instances, the molecular probe encoding a location of an analyte may associate with the analyte. In other cases, the molecular probe may associate with a derivative of the analyte. In some instances, the molecular probe encoding a location of an analyte may not associate with the analyte or a derivative of the analyte.

In some instances, the molecular probe encoding a location of an analyte may link to one region of the analyte. In other cases, the molecular probe encoding a location of an analyte may link to more than one region of the analyte. For example, a nucleic acid sequence encoding a location of an analyte may be designed to bind to multiple regions of another nucleic acid molecule.

In some instances, an analyte and a molecular probe encoding a location of the analyte may be processed or analyzed in the same reaction. For example, a first nucleic acid molecule and a second nucleic molecule encoding a location of the first nucleic acid molecule may be processed by a sequencing reaction. In other cases, an analyte and a molecular probe encoding a location of the analyte may be processed or analyzed in different reactions. For example, a first nucleic acid molecule may be processed by a sequencing reaction while a second nucleic acid molecule encoding a location of the first nucleic acid molecule may be processed by fluorescence hybridization and imaging.

The methods described herein may further comprise methods for attenuation or prevention of long-distance diffusion by reagents, such as by attenuating diffusion altogether or by attenuating diffusion along a certain direction(s) on the substrate, to retain spatial information of a sample (after diffusion begins). It may be undesirable for reagents to diffuse too far in a direction that is along an axis or plane contained in a final spatial map generated (e.g., x-y plane) as it may confuse proximity data that is later used to reconstruct the spatial map. The methods may prevent small particles that tend to diffuse relatively fast (e.g., DNA), compared to the duration of various reactions described herein (e.g., barcode release by USER enzyme, capture of analytes, etc.), from diffusing too far from an originating location before tagging of the analyte occurs, increasing the accuracy of a final spatial map. In some cases, diffusion can be attenuated by adding viscous reagents (e.g., PEG, etc.) and/or modulating one or more other reaction conditions (e.g., temperature).

In some cases, diffusion can be attenuated by encapsulating a reaction space in a gel, hydrogel (e.g., PEG hydrogel), or other mesh or matrix (e.g., polymer mesh matrix) to hinder particle movement therethrough. The encapsulation may be reversible. For example, the mesh or matrix may be degradable, such as after a certain period of time and/or upon application of one or more stimuli (e.g., chemical stimulus to induce, e.g., hydrolysis, enzymatic stimulus, photo stimulus, etc.). In an example, the reaction space comprising the sample and beads is crosslinked with a hydrophilic polymer to create a mesh that attenuates diffusion throughout the reaction space. The mesh may be nanoscale. In an example, a 4-arm PEG-acrylate macromer and PEG-dithiolglycolate crosslinker is used to form a PEG hydrogel that is degradable. In some cases, protein (e.g., bovine serum albumin (BSA) protein) or other solutes may be embedded or entrapped within the mesh network to increase a crowding effect to further attenuate diffusion. A pore size may be tuned by selecting component of changing length (molecular weight). Examples include tetra-acrylate PEG, cross-linked by thiol-PEG-thiol elements.

Alternatively or in addition, the reaction space may be subjected to electrophoresis to accelerate movement of charged particles (e.g., DNA, mRNA, spatial tags, etc.) along a direction of the electric field, such as along the z-axis when an x-y plane spatial map is generated, to attenuate diffusion along non-z-axis directions.

A probe as described elsewhere herein, or its derivative, may be enriched prior to amplification and/or sequencing. Enrichment may be facilitated by various capture mechanisms, such as a pair of a capture entity and a capturing entity. The capture entity may comprise or be biotin, a capture sequence (e.g., nucleic acid sequence) which may be hybridized to a strand of the probe or which may be part of another nucleic acid molecule conjugated to the probe, a magnetic particle capable of capture by application of a magnetic field, a charged particle capable of capture by application of an electric field, a combination thereof, or one or more other mechanisms configured for, or capable of, capture by a capturing entity. The capturing entity may comprise or be streptavidin when the capture moiety comprises biotin, a complementary capture sequence when the capture entity comprises a capture sequence, an apparatus, system, or device configured to apply a magnetic field when the capture entity comprises a magnetic particle, an apparatus, system, or device configured to apply an electrical field when the capture entity comprises a charged particle, a combination thereof, and/or one or more other mechanisms configured to capture the capture entity. In some instances, the capturing group may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, and the like). In some instances, the secondary capture entity can comprise a magnetic particle (e.g., magnetic bead) and the secondary capturing entity can comprise a magnetic system (e.g., magnet, apparatus, system, or device configured to apply a magnetic field, and the like). In some instances, the secondary capture entity can comprise a charged particle (e.g., charged bead carrying an electrical charge) and the secondary capturing entity can comprise an electrical system (e.g., magnet, apparatus, system, or device configured to apply an electric field, and the like). In some instances, the capture moiety comprises biotin, the capturing moiety comprises streptavidin coupled to a secondary capture entity, a magnetic bead, and the secondary capturing entity comprises a magnetic system. In situ capturing of analytes

In some instances, an analyte may be captured in situ for recording a location before further processing.

In some instances, an analyte may be detected by a probe. In some cases, detecting an analyte by a probe may comprise capturing the analyte by the probe. In some cases, capturing an analyte of a biological sample may comprise contacting the biological sample with a probe comprising a flow-based code domain comprising a nucleic acid sequence that encodes a flow-space sequence, a primer binding site; and a target-related domain. In some cases, a probe may comprise a flow-based code domain and encode a location of a captured analyte (e.g., location within a sample). In some instances, a target may be immobilized on a solid support. The solid support may comprise any of the substrates described elsewhere herein. The solid support, in some cases, may comprise a slide. In some cases, a slide may comprise an array. The slide may be a glass slide or be composed of other material. In some cases, the solid support may comprise a plurality of targets. For example, a slide may comprise a plurality of targets. Where the solid support is a slide, the slide may be integrated as a part of the substrate surface or otherwise coupled (e.g., as another layer) to the substrate. The substrate may be substantially planar, as described elsewhere herein.

In some instances, a location may comprise a coordinate in an absolute coordinate reference system. In some cases, a Cartesian coordinate system may be used as a coordinate for a target. In some cases, a Cartesian coordinate system may be used as a coordinate for an analyte. A Cartesian coordinate of an analyte, in some instances, may comprise an x and y coordinate on a two-dimensional plane. In some cases, a Cartesian coordinate of an analyte may comprise an x, y, and z coordinate on a three-dimensional plane. In some cases, a spatial coordinate may refer to the location on a solid support where a target is immobilized on. For example, in a two-dimensional image of an array slide comprising a plurality of targets, each spatial coordinate may represent each location where each target is located on the array slide.

In some instances, a molecular probe encoding a location may be attached to a substrate. In some cases, a molecular probe encoding a location may be immobilized on a substrate, such as directly onto the surface of the substrate, or via an intermediary medium such as a solid support. For example, the molecular probe may be immobilized to the solid support, which solid support is immobilized on the substrate. In some cases, the intermediary medium (e.g., solid support) may comprise a bead, a glass slide, an array slide, a gel bead, a magnetic bead, a microwell plate, or any combination thereof. An array slide, in some instances, may comprise a microarray slide. The substrate may comprise a plurality of individually addressable locations, as described elsewhere herein.

In some instances, a plurality of individually addressable locations may be smaller than the biological sample being analyzed such as that information extracted from the plurality of individually addressable locations may be used to identify one sample. In some cases, a biological sample bigger than one substrate comprising a plurality of individually addressable locations may be analyzed by more than one such substrate for analysis. For example, at least two substrates, each comprising a plurality of individually addressable locations, may be required to cover a biological sample comprising a plurality of analytes. Each substrate may contact a non-overlapping or overlapping section of the biological sample, contacting or capturing a non-overlapping or overlapping subset of analytes. The result obtained from the two substrates may be combined to reconstruct the spatial location of each analyte in the biological sample.

In some instances, a molecular probe encoding a location may be attached to an individually addressable location on a substrate. In some instances, a probe comprising a flow-based code domain may be attached to an individually addressable location on a substrate. In some instances, a probe comprising a flow-based code domain may be attached or immobilized to an individually addressable location on a substrate. In some cases, a first probe comprising a first flow-based code domain may be attached or immobilized to a first individually addressable location while a secondary probe comprising a second unique flow-based code domain may be attached or immobilized to a second individually addressable location on a substrate. In some instances, a substrate may be planar. The surface of a substrate may be substantially planar. The surface of the substrate may be patterned or textured, such as to comprise a plurality of wells. The surface of a substrate, in some cases, may also comprise any cross-sectional surface profiles described herein, such as but not limited to those in FIGS. 3A-3G. Patterning of the surface of a substrate, in some instances, may comprise surface chemistry. In some cases, any surface chemistry described herein may be used to pattern the surface of a substrate.

In some instances, a substrate may have any shape as described herein. In some cases, a shape of a substrate may comprise a regular polygon or an irregular polygon. A polygon, in some cases, may comprise a triangle, square, rectangle, quadrilateral, pentagon, hexagon, heptagon, octagon, nonagon, or decagon. A polygon, in other cases, may comprise a number of sides. A number of sides for a polygon, in some cases, is 3, 4, 5, 6, 7, 8, 9, 10, 11, 12, 13, 14, 15, 16, 17, 18, 19, or 20. In some cases, a shape of a substrate may be an ellipse. An ellipse, in some cases, may comprise a circle or an oval.

In some instances, each flow-based code domain of a probe may comprise a unique molecular identity. For example, each flow-based code domain may comprise a nucleic acid sequence encoding a unique flow-space sequence. Each unique flow-space sequence may allow each flow-based code domain to be distinguished from other flow-based code domains. In some cases, the identity of each flow-based code domain is pre-determined as the probe is immobilized. In other cases, the identity of each flow-based code domain is determined after the probe is immobilized. Other identities based on unique physical, chemical, or biological attributes may also be used as a molecular identity.

In some instances, a substrate may comprise a plurality of sets of probes, each detecting one type of analytes. For example, a substrate may comprise both oligo(dT) primer sequence probes to capture endogenous mRNA sequences and nucleosome-targeting sequence probes to capture nucleosomes. In some cases, probe sequences targeting different types of analytes with the same location may have the same flow-space sequence.

In some instances, a substrate may comprise a cluster of probes encoding flow-space sequences for each type of probe. In some cases, a cluster of probes encoding flow-space sequences may comprise about 1×10⁰ molecules, about 1×10¹ molecules, about 1×10² molecules, about 1×10³ molecules, about 1×10⁴ molecules, about 1×10⁵ molecules, about 1×10⁶ molecules, about 1×10⁷ molecules, about 1×10⁸ molecules, about 1×10⁹ molecules, about 1×10¹⁰ molecules, about 1×10¹¹ molecules, about 1×10¹² molecules, about 1×10¹³ molecules, about 1×10¹⁴ molecules, about 1×10¹⁵ molecules, about 1×10¹⁶ molecules, about 1×10¹⁷ molecules, about 1×10¹⁸ molecules, about 1×10¹⁹ molecules, or about 1×10²⁰ molecules.

In some instances, a biological sample is treated by a fixative, or fixated, before contacting the probe. Fixation, in some cases, may render the location of an analyte invariable. For example, an mRNA molecule may not diffuse away from its location in a tissue after fixation. Contacting the fixed tissue to a substrate (e.g., an array slide, wafer, etc.) with a plurality of probes may allow the probe to capture the endogenous mRNA at its fixed location in the tissue. In some cases, a permeabilization step of a fixed biological sample may facilitate the contacting between the probe and the analyte. In some cases, a permeabilization of a biological sample may release an analyte. For example, permeabilization of a fixed tissue may release an mRNA vertically downward, e.g., via gravity, so that it can contact the probe. Once captured, the endogenous mRNA is encoded with the location information encoded by the flow-space sequence.

In some instances, a biological sample may be contacted with more than one set of probes, wherein each set of probes is immobilized on a different substrate or different regions of a same substrate. In some cases, a biological sample may be contacted with two sets of probes. The two sets of probes may detect or capture the same analyte. In some cases, detecting the same analyte by two sets of probes may facilitate an analysis of the analyte. For example, an analyte may be assigned to an addressable location with higher confidence level if the analyte is detected by two sets of probes assigned to the same addressable location on two different substrates (or to corresponding addressable locations on two different regions of a same substrate), compared to an analyte that is only detected by one set of probes. In some instances, two sets of probes of two different substrates assigned to the same addressable location (or same corresponding addressable locations) may detect or capture different analytes. For example, a first substrate or first substrate region may comprise oligo(dT) primer sequence probes to capture endogenous mRNA sequences. A second substrate or second substrate region may comprise nucleosome-targeting sequence probes to capture nucleosomes. In some cases, probe sequences targeting different types of analytes with the same location may encode for the same flow-space sequence.

In some instances, a probe linked to an analyte may be released from a solid substrate. In some cases, a probe may be cleavable or otherwise releasable. In some cases, a probe may comprise a cleavable linker. In some cases, a probe may be cleavable by a stimulus (e.g., physical or chemical stimulus). The probe may be released prior to, during, or subsequent to binding to an analyte. In some cases, once a probe binds to an analyte(s), the probe may be released from the solid substrate by cleaving the probe. In some cases, a probe may be released from a solid substrate once the probe has a link with or otherwise encodes the spatial information location to an analyte. In some cases, a captured analyte may be released from a probe once the analyte is encoded with a spatial location. For example, a bead comprising a probe that encodes spatial location (e.g., via a flow-space sequence encoded in a flow-based code domain) may be contacted with an analyte to link the probe with the analyte (e.g., a target nucleic acid may hybridize to the probe), and optionally the complex may be further processed (e.g., subjected to an extension reaction), after which the probe-analyte complex, or derivative thereof, is cleaved from the substrate (e.g., wafer, bead, etc.).

In some instances, a probe may be used in a downstream reaction after capturing an analyte. For example, an oligo(dT) primer sequence may become a primer for a reverse transcription reaction once it captures an mRNA molecule. In some instances, a downstream reaction may not proceed or be triggered until a reagent is supplied. In some cases, a reagent to trigger a downstream reaction may be supplied by the systems and methods described herein or thereof. For example, a reverse transcription may not proceed after the oligo(dT) sequence captures an mRNA molecule until the nucleotides, enzymes, buffers, or any combination thereof are provided to the hybridized oligo(dT)-mRNA sequences. In some instances, a downstream reaction may create derivatives of an analyte. Such derivatives may facilitate other processing, such as but not limited to, an amplification or sequencing reaction. For example, an amplification may comprise a reverse transcription, primer extension, PCR, LCR, helicase-dependent amplification, asymmetric amplification, RCA, RPA, LAMP, NASBA, 3SR, HCR, MDA, derivatives thereof, or any combination thereof. An amplification may also be a linear or non-linear amplification. In one case, a reverse transcription of an mRNA molecule may create a cDNA molecule, wherein the cDNA molecule may be amplified, e.g., by PCR or RCA, and sequenced by a sequencer. In some cases, a sequencing reaction may be carried out using flow-based sequencing as described herein (e.g., Examples).

In some instances, a biological sample may be dissected, dissociated, digested, or degraded after an analyte is encoded with the location information. In some cases, a biological sample may be dissected, dissociated, digested, or degraded after a probe captures an analyte and a flow-space sequence is linked to the analyte. Dissection, dissociation, digestion, or degradation of a biological sample may facilitate the processing of the processing of an analyte. Once linked with a flow-space sequence, in some cases, the location of an analyte may be retained and decoded afterwards. For example, dissection, dissociation, digestion, or degradation of a biological sample may not remove the encoded location information of an analyte. The location of each analyte may be reconstructed digitally by decoding the flow-space sequence of the analyte.

In some instances, a biological sample may be aligned with the contacted solid support with a plurality of probes encoding flow-space sequences to facilitate the mapping of the analytes to their locations in the biological sample. For example, a tissue is contacted with a substrate comprising a plurality of probes comprising oligo(dT) capture primers and encoding flow-space sequences, arranged in a rectangular grid, to capture the mRNA molecules. The tissue is then imaged to align the rectangular grid on the tissue. Since the location and sequence of each flow-space sequence on the rectangular grid is known, the location of the mRNA molecules in the tissue can be identified by decoding the flow-space sequences and overlaying them on the imaged tissue with the rectangular grid.

In some instances, a probe may be attached to beads. In some cases, probes attached to beads may be dispensed on a substrate (e.g., a wafer, a glass slide, or a microwell plate). The probe-bound beads may be dispensed in a similar manner as sample-bound beads are dispensed, as described elsewhere herein. In some cases, dispensing probes attached to beads on a solid support may facilitate an increase of the density of the probes compared to that when the probe is attached directly to a slide or glass slide. Such an increase in density may be 1-fold, 10-fold, 100-fold, 100-fold, 1,000-fold, 10,000-fold, or more.

In some instances, dispensing beads with probes on a substrate may create a random distribution of probes encoding different flow-space sequences on the solid support. In some instances, beads comprising probes encoding different flow-space sequences may be deposited into microwells of a microwell plate. In some cases, the addressable location of a flow-space sequence of a probe may not be pre-determined when a bead comprising the probe is dispensed or deposited. In some cases, the addressable location of a flow-space sequence of a probe may be determined once a bead comprising the probe is dispensed on a substrate. For example, the flow-space sequence may be identified by flow-based sequencing as described herein (e.g., Examples).

In some instances, at least about 100 sections may be processed simultaneously by systems and methods disclosed thereof. In some cases, at least about 200 sections may be processed simultaneously by systems and methods disclosed thereof. In some cases, at least about 300 sections may be processed simultaneously by systems and methods disclosed thereof. In some cases, at least about 400 sections may be processed simultaneously by systems and methods disclosed thereof. In some cases, at least about 500 sections may be processed simultaneously by systems and methods disclosed thereof. In some cases, at least about 600 sections may be processed simultaneously by systems and methods disclosed thereof. In some cases, at least about 700 sections may be processed simultaneously by systems and methods disclosed thereof. In some cases, at least about 800 sections may be processed simultaneously by systems and methods disclosed thereof. In some cases, at least about 900 sections may be processed simultaneously by systems and methods disclosed thereof. In some cases, at least about 1000 sections may be processed simultaneously by systems and methods disclosed thereof. In some cases, at least about 1100 sections may be processed simultaneously by systems and methods disclosed thereof. In some cases, at least about 1200 sections may be processed simultaneously by systems and methods disclosed thereof. In some cases, at least about 1300 sections may be processed simultaneously by systems and methods disclosed thereof. In some cases, at least about 1400 sections may be processed simultaneously by systems and methods disclosed thereof. In some cases, at least about 1500 sections may be processed simultaneously by systems and methods disclosed thereof. In some cases, at least about 1600 sections may be processed simultaneously by systems and methods disclosed thereof. In some cases, at least about 1700 sections may be processed simultaneously by systems and methods disclosed thereof. In some cases, at least about 1800 sections may be processed simultaneously by systems and methods disclosed thereof. In some cases, at least about 1900 sections may be processed simultaneously by systems and methods disclosed thereof. In some cases, at least about 2000 sections may be processed simultaneously by systems and methods disclosed thereof. In some cases, at least about 2100 sections may be processed simultaneously by systems and methods disclosed thereof. In some cases, at least about 2200 sections may be processed simultaneously by systems and methods disclosed thereof. In some cases, at least about 2300 sections may be processed simultaneously by systems and methods disclosed thereof. In some cases, at least about 2400 sections may be processed simultaneously by systems and methods disclosed thereof. In some cases, at least about 2500 sections may be processed simultaneously by systems and methods disclosed thereof. In some cases, at least about 2600 sections may be processed simultaneously by systems and methods disclosed thereof. In some cases, at least about 2700 sections may be processed simultaneously by systems and methods disclosed thereof. In some cases, at least about 2800 sections may be processed simultaneously by systems and methods disclosed thereof. In some cases, at least about 2900 sections may be processed simultaneously by systems and methods disclosed thereof. In some cases, at least about 3000 sections may be processed simultaneously by systems and methods disclosed thereof. In some cases, systems and methods disclosed herein can process dozens, hundreds, or thousands of standard tissue sections (e.g., at the same time, including but not limited to about 20, 50, 100, 200, 300, 500, 600, 800, 1,000, 1,500, 2,000, 3,000, 5,000 7,000, 10,000 or more. In some embodiments, tens of thousands or more tissue sections can be processed. A standard tissue section can be of a size that is comparable to tissue size founded in conventional Formalin-Fixed, Paraffin-Embedded (FFPE) tissue slides.

In some cases, a plurality of sections of a substrate may be reconstructed. Reconstruction of the substrates, in some cases, may reconstruct a biological sample. The reconstructed biological sample may retain the same biological context of the biological sample before the reconstruction. Such a biological context, in some cases, may comprise the composition of the biological sample, the location of each constituent of the biological sample, the identity of each constituent of the biological sample, or any combinations thereof. For example, a tumor sample comprising a heterogeneity of cancer cell types may undergo the fluorescent image analysis described in this disclosure. Each cell or subset of cells of the tumor sample may be processed by the fluorescent image analysis simultaneously. After the fluorescent image analysis, the analyzed cells or subsets of cells of the tumor may be reconstructed to represent the cellular context of the tumor, i.e., the heterogeneity of the cancer cell types of the tumor sample. In other cases, a biological sample may comprise more than one tissue. For example, a tumor sample may comprise cancer cell and normal cells. In other cases, a tumor sample may comprise a tumor and a normal tissue.

In some instances, a biological sample may be processed while on a substrate. In some cases, a biological sample may be processed while immobilized on a substrate. In some cases, a reagent may be dispensed over the biological sample using the systems described in this disclosed. For example, a reagent for permeabilization may be dispensed on a tissue on an immobilized substrate. The cells in the tissue may then be lysed on the substrate. In other cases, other reagents, such as those for sequencing; or other described methods that can identify the spatial flow-based sequence of an analyte, the sections of the substrate, the sub-samples of a biological sample, or the cells of the biological sample may also be dispensed on the substrate or biological sample. Beneficially, the on-substrate processing can make reagent distribution highly efficient, and obviate the need to transfer a sample or analyte from one container (or location) to another container (or location). In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 addressable locations of substrate. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 analytes of a substrate. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 analytes of a biological sample. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 sections of a substrate. In some cases, a reagent may be dispensed over at least about 10, at least about 50, at least about 100, at least about 500, at least about 1000, at least about 5000, at least about 10000, at least about 50000, at least about 100000, at least about 500000, at least about 1000000, at least about 5000000, at least about 10000000, at least about 50000000 cells of a tissue.

In some instances, the systems and methods disclosed herein may quantify the number of a plurality of analytes. In other cases, the systems and methods disclosed herein may quantify the number of a plurality of cells. In some cases, the systems and methods disclosed herein also may the number of a plurality of sub-samples or sections. In situ sequencing

In some instances, an analyte or its derivative of a biological sample may be screened while it remains in its endogenous context or structure of the biological sample. In some cases, an analyte or its derivative of a biological sample may be screened without being dissociated from its endogenous biological context or structure in the biological sample. In some cases, an analyte or its derivative of a biological sample may be screened while other molecules in the biological sample are removed from the same biological context or structure in the biological sample. For example, a nucleic acid that encodes a flow-space sequence, or another nucleic acid molecule or its derivative, may be sequenced while it is still attached to the tissue where it originates from. In some instances, screening an analyte or its derivative of a biological sample while it remains in its endogenous biological context or structure may retain the location of the analyte of the biological sample.

In some instances, an analyte may be converted to a derivative. In other cases, an analyte may be converted to a series of derivatives. In some cases, an analyte may be converted to a first derivative, and the first derivative may then be converted to a second derivative. For example, an RNA molecule may be converted to a cDNA molecule via a reverse transcription. In another example, an RNA molecule may be converted to a cDNA molecule via a reverse transcription, and the cDNA molecule may be converted to an RCA product via an RNA amplification.

In some instances, a downstream reaction may comprise an amplification reaction. In some cases, a downstream reaction may comprise a nucleic acid amplification reaction. For example, an amplification may comprise a reverse transcription, primer extension, PCR, LCR, helicase-dependent amplification, asymmetric amplification, RCA, RPA, LAMP, NASBA, 3SR, HCR, MDA, derivatives thereof, or any combination thereof. An amplification, in some instances, may also be a linear or non-linear amplification. In some cases, a nucleic acid analyte may be amplified to form nanoballs for further processing. A nanoball, in some cases, may be single stranded or double stranded.

In some instances, an analyte or its first derivative may be modified to a second derivative during a conversion reaction described herein. In some cases, a target nucleic acid molecule or its first derivative may be modified to a second derivative during an amplification reaction. One such modification may comprise a chemically modified base (e.g., an amine-modified base) during the amplification. In some cases, a modification to a second derivative may facilitate embedding of the second derivative to the endogenous context or structure of its original target nucleic acid molecule. In some cases, a modification to a second derivative may facilitate a cross-linking of the second derivative to the cellular protein matrix. In some cases, amine-based modification may link in situ synthesized second derivative to a cellular polymer at the endogenous location of its original target nucleic acid molecule. In some cases, a modification to a second derivative may also facilitate removal of other undesirable molecules. Such undesirable molecule may comprise any molecules other than the second derivative (e.g., proteins or lipids). In some instances, an analyte may be removed from a biological sample or degraded after being processed. In some cases, an analyte may be removed from a biological sample or degraded after being converted to a derivative. For example, an RNA molecule may be removed after it is converted to a cDNA molecule via a reverse transcription reaction. In other cases, a first derivative of an analyte may be removed from a biological sample or degraded after being modified to a second derivative. In some cases, an analyte may be removed from a biological sample or degraded after being converted to a derivative. In some cases, even after an analyte or a first derivative of the analyte is removed, a subsequent derivative of the analyte or the first derivative may retain the endogenous location of the analyte.

In some instances, an analyte or its derivative in a biological sample may be determined by a flow-based sequencing reaction using methods and systems as described herein (e.g., Examples) while the analyte or the derivate remains in its endogenous location the biological sample. For example, a flow-space sequence encoded by a probe bound to the analyte may be sequenced. In some cases, an analyte or its derivative in a biological sample may also be determined by sequencing-by-synthesis (SBS) or its derivatives while the analyte or the derivate remains in its endogenous location the biological sample. In some cases, a biological sample may be processed to allow flow-based sequencing using methods and systems as described herein (e.g., Examples) while the analyte or the derivate remains in its endogenous location the biological sample.

### Probe Design

A probe of the present disclosure may comprise a (1) target-related domain, (2) primer binding site, and (3) flow-based code domain, in any useful order.

In some cases, the probe comprises a flow-based code domain comprising a nucleic acid sequence encoding a flow-space sequence, a first primer binding site; and a first target-related domain.

The domains within the probe may be spaced with linkers or spacer sequences composed of either additional nucleotides or other molecules such as PEG. Different spacer sequences within a probe may be the same sequence or different sequences. Spacer sequences may comprise a sequence of any length. For example, the spacer can be any internal spacer, e.g., C3 spacer, C12 spacer, spacer 9, spacer 18, etc. In some cases, the spacer sequence may be designed to not be complementary to any probe sequence, or portion thereof. In some cases, the primer binding site and/or flow-based coding domain are repeated multiple times along the same probe in order to increase the intensity of the measured signal.

In some cases, the flow-based code domain may be positioned 5′ to the first primer binding site, and the first primer binding site may be positioned 5′ to the first target-related domain. In some cases, the probe may further comprise a first PCR primer binding site positioned 5′ to the flow-based code domain or 3′ to the first target-related domain. In some cases, the probe may further comprise a bead adapter 3′ to the first target-related domain. In some cases, the probe may further comprise a sequencing primer hybridized to the first primer binding site. In some cases, the sequencing primer may further comprise a second target-related domain and/or a linker. In some cases, the linker may be positioned 5′ to the sequencing primer and the second target-related domain may be positioned 5′ to the linker. In some cases, the first target-related domain and the second target-related domain are configured to bind to two different targets.

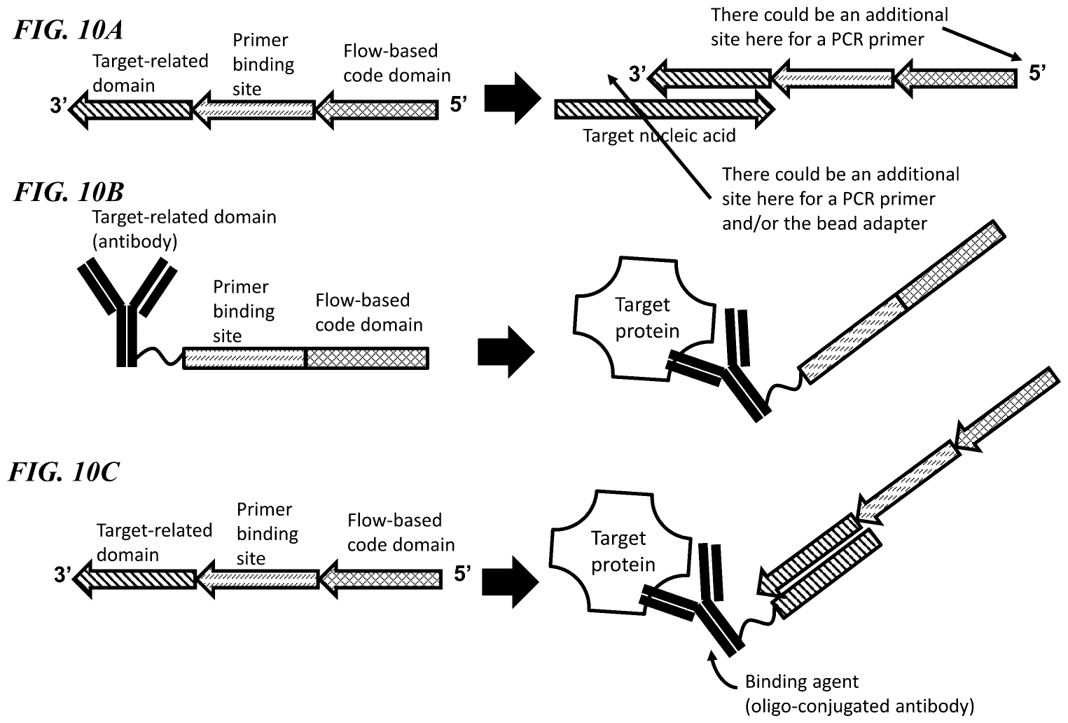

FIG. 10 illustrates exemplary probes that include a flow-based code domain. A probe including a flow-based code domain may be sequenced using flow-based sequencing.

In some cases, the probe may comprise a different order of the functional sequences other than that shown in FIG. 10. For example, the probe may comprise in order, the primer binding site, the flow-based code domain, and the target-related domain.

The target-related domain may bind to a target directly or indirectly. The target-related domain may comprise an oligonucleotide, a nucleic acid molecule, an aptamer, an antibody or binding fragment thereof, affinity binding protein, lipid, carbohydrate, or any combination thereof. In some instances, the target-related domain comprises an oligonucleotide, an aptamer, an antibody or binding fragment thereof, or any combination thereof. The target-related domain may be configured to bind to a target that is an analyte. An analyte may be or comprise any analyte described herein. In some instances, an analyte may comprise a nucleic acid, polypeptide, protein, antibody or binding fragment thereof, metabolite, carbohydrate, lipid, derivatives thereof, or any combinations thereof. A nucleic acid may be or comprise any nucleic acid molecule described herein. In some cases, a nucleic acid may comprise deoxyribonucleic acid (DNA), including genomic DNA), ribonucleic acid (RNA), locked nucleic acid (LNA), peptide nucleic acid (PNA), modified nucleic acid (XNA), derivative thereof, or any combination thereof. In some cases, a nucleic acid may comprise an endogenous or exogenous chemical modification. Such a modification may comprise a sugar modification, a base modification, a backbone modification, or an unnatural base pairing. For example, DNA may be methylated or acetylated. In some cases, a carbohydrate may comprise a monosaccharide, a polysaccharide, a lignin, derivatives thereof, or any combinations thereof. In some cases, a peptide or a protein may be modified. A modification of a protein or a peptide, in some cases, may comprise an ubiquitylation, sumolyation, ubiquitin-like modification, phosphorylation, methylation, acetylation, proteolysis, glycosylation, isoprenylation, deamidation, eliminylation, AMPlyation, ADP-ribosylation, redox, any derivatives thereof, or any combinations thereof. In some cases the analyte may comprise an antibody or binding fragment thereof, an oligonucleotide, an RNA transcript, a protein, a polypeptide, a metabolite, genomic DNA, or any combination thereof.

The target-related domain sequence may be configured to capture (or bind to) an analyte, such as an RNA molecule. For example, the target-related domain may comprise a polyT sequence configured to capture a polyA tail sequence of an mRNA molecule. In another example, the target-related domain may comprise a random sequence, a targeted sequence, or any other sequence designed to bind to an analyte sequence, or derivative thereof. In some cases, the target-related domain may comprise a random n-mer sequence. In some examples, for targeted mRNA assays, the target-related domain may comprise a target mRNA sequence (or derivative thereof). In some examples, for targeted genomic DNA (gDNA) assays, the target-related domain may comprise a target gDNA sequence (or derivative thereof). In some examples, for antibody assays, the target-related domain may comprise a sequence configured to capture an oligonucleotide conjugated to one or more antibodies (e.g., DNA capture tags), or a derivative thereof. In some examples, for assays that utilize template switching reactions, the target-related domain may comprise a sequence configured to bind to a product of a reverse transcription reaction, such as a polyG sequence. In some examples, for assays that utilize one or more probes, the target-related domain may comprise a sequence corresponding to a sequence of the probe, to a molecule associated with the probe, or derivative thereof. In some examples, the target-related domain may be part of a single strand portion, a double strand portion, or partially double-stranded complex. In some examples, the target-related domain may be part of a hybrid DNA/RNA complex. In some examples, for a transposition assay concerning gDNA analytes, after a transposition reaction (e.g., subsequent to Tn5 transposase treatment of gDNA, where the Tn5 transposase comprises one or more barcode and/or adapter sequences), a partially double-stranded analyte may be generated. In some examples, at least one end of the partially double-stranded analyte may comprise an overhang comprising a barcode and/or adapter sequence. A target-related domain may be configured to capture the overhang of the partially double-stranded analyte comprising the barcode and/or adapter sequence. One or more gap filling and/or ligation reactions may be performed to join the partially double-stranded bridge construct and transposition analyte.

For example, as shown in FIG. 10 panel A, the target-related domain may comprise a target nucleic acid sequence that directly binds to target DNA or RNA. In another example, as shown in FIG. 10 panel B, the target-related domain may comprise an antibody that directly binds to a target protein. In another example, as shown in FIG. 10 panel C, the target-related domain may comprise a nucleic acid sequence that binds to another binding agent (also referred to as affinity agent), such as to an oligonucleotide in an oligonucleotide-conjugated antibody, which binding agent binds to the target analyte (e.g., target protein). For a single assay, there may be one type of target analyte or multiple types of target analytes. In the latter case, a probe set may be designed to include multiple types of target-related domains.

The primer binding site may allow the probe, or its derivative (e.g., complement, amplicon, or the like), to bind to a sequencing primer to initiate a primer extension reaction for sequencing. In some cases, the primer binding site may be disposed 5′ of the target-related domain and 3′ of the flow-based code domain (e.g., between the target-related domain and the flow-based code domain). This may allow skipping through sequencing of the target-related domain, as only the flow-based code domain is needed to determine the identity of the target.

The flow-based code domain may comprise a nucleic acid sequence encoding a “flow-space sequence”, which may be a nucleic acid sequence which encodes the identity of the probe, or its target analyte, at a “key flow position” (otherwise known as “mostly 0 flow” or “interrogation flow”) when the probe is sequenced using flow-based sequencing. As described above, flow-based sequencing is a type of sequencing-by-synthesis (SBS) where (1) the templates are hybridized to sequencing primers on a substrate, and (2) the sequencing primers are extended with a series of non-terminated, single-base nucleotide flows in a specific flow order, where after each flow, the substrate is washed to remove unincorporated nucleotides, the substrate is imaged to detect incorporation events, and the labels are cleaved prior to the next flow. The flow-space sequence for a particular nucleic acid sequence may be the sequence of integers that represent the relative signals measured for a sequence of flows interrogating the particular nucleic acid sequence in a specific flow order. In flow-based sequencing, a nucleic acid base sequence can be represented as a flow-space sequence tied to the specific flow order, the flow-space sequence comprising a sequence of integers that represents the signal readout at each flow position. A flow-space sequence may be represented as a flow chart or flowgram in a one-dimensional matrix or linear array. As a simple example, using the specific flow order [T G C A] in flow-based sequencing, the nucleic acid base sequences of “ACT” and “AGT” will yield the flow charts [1 1 0 1] and [1 0 1 1], respectively. In the present disclosure, a set of probes may be designed to comprise respective flow-based code domains where the respective identities of the individual probes, or their target analytes, are encoded across different key flow positions in the flow chart when the probes are sequenced using flow-based sequencing. With this flow-based barcoding design, it is possible to identify the presence or absence, and optionally the location, of a different probe with/after each flow. In some cases, this may provide the ability to deduce the identity of the probe from a few flows as opposed to from the full length of the sequence. Thus, a flow-space sequence of a probe may be configured to generate a flowgram unique to the probe amongst a plurality of probes during flow-based sequencing. In some cases, the flowgram comprises a set of relative intensity values generated during the flow-based sequencing.

### Orthogonal Encoding

In some cases, the flow-based code domain may have an orthogonal encoding design, where a key flow position is encoded to be ‘on’ (produce any homopolymer signal ‘H’) or ‘off’ (produce no signal, ‘0’). In some cases, the flow-based sequence comprises a key flow position every (3n−1)^{th} and (3n)^{th} flow positions, where n is a positive integer. That is, each probe comprises a flow-based code domain having the following same flow chart: [1 0 0 1 0 0 1 0 0 1 0 0 1 . . . ], except each probe is encoded with a ‘H’ not a ‘0’ at a different (3n−1)^{th} or (3n)^{th} flow position. The ‘key flow’ position is also referred to herein as ‘mostly 0 flow’ position or ‘interrogation flow’ position. The following lists out different probes in an example probe set with their flow charts:
Probe 1: [1 H 0 1 0 0 1 0 0 1 0 0 1 . . . ];
Probe 2: [1 0H 1 0 0 1 0 0 1 0 0 1 . . . ];
Probe 3: [1 0 0 1 H 0 1 0 0 1 0 0 1 . . . ];
Probe 4: [1 0 0 1 0 H 1 0 0 1 0 0 1 . . . ];
Probe 5: [1 0 0 1 0 0 1 H 0 1 0 0 1 . . . ], and so forth, where each ‘H’ refers to the respective homopolymer length of the probe.

Thus, within a total of [x] flows, a total of (⅔)x unique target analytes may be encoded for. It is possible for ‘H’ to vary amongst different probes in a probe set. In the above example, e.g., H=6 for Probe 1, H=8 for Probe 2, H=9 for Probe 3, etc. In some cases, the H for each probe in a probe set can be specifically designed to match the dynamic range of high- and low-abundance targets. For example, if Target 1 (targeted by Probe 1) is generally 10 times more abundant than Target 2 (targeted by Probe 2), H for Probe 1 can be selected to be a relatively lower number than the H for Probe 2. In some cases, the H for each probe may be designed with practical implications in mind (e.g., longer T homopolymers can be synthesized compared to longer G homopolymers).

It will be appreciated that while the above examples show that a single key flow position is encoded to form a unique flow chart amongst the probe set, multiple key flow positions may be encoded to form a unique flow chart in the probe set. In the following example:
Probe 1: [1 0 0 1 1 1 1 0 0 1 . . . ]; and
Probe 2: [1 0 0 1 1 0 1 1 0 1 . . . ],
   a signal at a key flow position can be decomposed to the constituent N non-linearly dependent vectors (for respective N probes, where N corresponds to the number of flows). In some cases, encoding multiple key flow positions for a probe may be more robust (i.e., less prone to error) than encoding a single key flow position, as the identification of that probe is not dependent on a single flow.

In some case, it is possible to encode information in all of the flow positions, not just the key flow positions (which is ⅔ of the total flows). For example:
Probe 1: [1 1 0 0 1 2 1 . . . ]; and
Probe 2: [1 0 2 1 0 0 2 . . . ].

In this case, a composite signal can be decomposed into N vectors, which may be dependent on if any signal noise is successfully distinguished and filtered out.

A probe set may be designed to have as many key flow positions for encoding as possible. To put in another way, in order to maximize the number of interrogation flows, probes may be designed to encode flow-space sequences that include as many zeros as possible. Flow-space sequences may not, by definition, have 3 consecutive zeros, so the sequence of a given length with the maximal number of zeros and lowest number of bases is 1001001001 [ . . . ], where ⅔ of the flows are 0. Non-key flow position readouts (otherwise known as “non-interrogation flows”) may not be informative to distinguish different probes from each other, but may still serve a useful purpose, such as used to resolve the shape of the sample, detect single molecules or concentrations of molecule, control for variations in the signal across space and time (e.g. droop, phasing, illumination pattern, and the like).

For the probes to work better in practice, they may be designed so that they do not form a strong secondary structure; otherwise, synthesizing their reverse complement strand may be challenging. By checking six simple flow orders of four bases starting with a “T” flow, two flow orders best accomplish this: TGAC and TCAG, as shown in Table 2. For TGAC, the repeat element in the flow-based code domain is TCAG, and for TCAG the repeat element is TGAC. Other flow order and flow-space sequences are possible but may yield a lower number of interrogation flows.

TABLE 2

| | Ideal Flow-based coding | |
|---|---|---|
| | domain repeat element | Ideal secondary structure design |
| Flow Order | (1001001001) | (no strong secondary structure) |
| TGCA | TACG | No |
| TGAC | TCAG | Yes |
| TCAG | TGAC | Yes |
| TCGA | TAGC | No |
| TACG | TGCA | No |
| TAGC | TCGA | No |

In order to increase the measured signal intensity, the probes may include long nucleic acid base homopolymers, for example:
Probe 1—1901001
Probe 2—1091001
Probe 3—1001901
Probe 4—1001091

Nucleic acid base homopolymers may also be longer than 9 bases. Additionally, once a probe reaches its interrogation flow, its signal can be terminated, for example:
Probe 1—19
Probe 2—109
Probe 3—10019
Probe 4—100109

Multiple probes or probe pairs can be designed to multiple regions/epitopes in the target to increase signal intensity.

In cases where individual molecules may be resolved separately in the imaging system, a combinatorial coding scheme may be used that allows for a larger number of probes to be resolved. In this scheme, every interrogation flow is treated as a digit that is either binary (zero vs non-zero, or 0-mer vs another H-mer) or of higher order (e.g., 0/4/8/12 mers encoding a base 4). All the flows may be used together to encode all the probes in an assay to maximize information, or the probes may be divided into groups that are assigned non-overlapping interrogation flows in order to reduce potential crosstalk and spatial overlap between probes. More rigorously, a group of M targets may be considered, where an encoding of base B is used (e.g., B=2 for binary coding). The target can be divided into groups of MBⁿ, where n is the number of interrogation flows assigned to each group. For example, with n=5 and B=2, M=32 targets may be encoded by flow-based coding domains. Thus, with 32 targets of n=5 interrogation flows each, using a total of 270 flows, 180 of which are interrogation flows, 1152 flow-based coding domains may be encoded [(180 total interrogation flows/5 interrogation flows)*(32)=1152].

In some circumstances, to avoid potential crosstalk between probes (e.g., misidentification of one probe for another), probes may be sparsely encoded. That is, a subset of probe sequences may be selected where each probe is sufficiently different from the other probes such that little or no signal deconvolution is required to distinguish probe sequences. For instance, with n=24 (24 interrogation flows) and B=2, there will be a set of 16,777,216 (=2²⁴) possible probes; however, a subset of those probes could be selected such that each pair of probes in the subset has at least 3 changes (e.g., an edit distance) between each other, which would result in a subset of 1213 (approximately=(2²⁴)/(24³))possible probes.

An edit distance may be calculated using a variety of approaches. In some instances, the edit distance can be calculated by counting (e.g., using the at least one processor) a number of different elements between two probes. The edit distance may be any useful edit distance (e.g., a Levenshtein distance, a longest common subsequence distance, a Hamming distance, a Jardo distance, a Damerau-Levenshtein distance, or analogs or derivatives thereof).

As one example, a Hamming distance may be calculated for all pairs of probes in a set (e.g., to select a subset of probes that differ by at least an edit distance threshold). In such an example, for any given pair of probes, each position (e.g., element, which may comprise a flow cycle value, e.g., 0, 1, or H) of the first probe of the pair is compared to the corresponding position in the second probe of the pair. If the values differ for a given position, a value of 1 distance unit is added (e.g., every position in the pair of probes that differs increases the value of the edit distance between the pair of probes by 1). By way of example, a probe pair with a first probe comprising 1901001 and a second probe comprising 1091001, has an edit distance of 2, as two positions (the second and third elements) differ in value. Each position in the pair of probes that does not differ in value (e.g., the first, fourth, fifth, sixth, and seventh elements in this example) does not increment the edit distance.

In another example, probes in the following subset of probes, from a probe set where n=5 (5 interrogation flows), each have an edit distance of at least 3 from each other:
Probe A: 11000
Probe B: 00110
Probe C: 01001
Probe D: 10010

The edit distance threshold between all pairs of probes may be any useful value. In some instances, a higher edit distance threshold may be applied in order to increase the resulting signal distinction between probes. The edit distance threshold may be at least 1, at least 2, at least 3, at least 4, at least 5, at least 6, at least 7, at least 8, at least 9, at least 10 distance units, or more. Alternatively or in addition, the edit distance threshold may be at most 10, at most 9, at most 8, at most 7, at most 6, at most 5, at most 4, at most 3, at most 2, or at most 1 distance units.

In some cases, multiplexing may be increased by including multiple types of sequencing primers and primer binding sites. In some cases, only one sequencing primer may be active and the other sequencing primers and/or primer binding sites may be blocked with a blocking group. Once the probes matching the first primer are sequenced through, the second primer may be activated by cleaving at a cleavage site to remove a blocking group. In some cases, additional primers may be activated one by one, based on varying blocking methods.

The cleavage site may comprise one or more cleavable moieties. In some cases, the blocking moiety may comprise a cleavable moiety. A cleavable moiety may comprise any useful cleavable or excisable moiety that can be used to cleave an oligonucleotide (or portion thereof). For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethylene glycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some cases, the additional primer binding site and/or sequencing primer may comprise a reversible terminator (e.g., blocking group) configured to terminate polymerase reactions (until unblocked). For example, a nucleotide may comprise a reversible terminator, or a moiety that is capable of terminating primer extension reversibly. The blocking moiety may comprise a dideoxynucleotide. Nucleotides comprising reversible terminators may be accepted by polymerases and incorporated into growing nucleic acid sequences analogously to non-reversibly terminated nucleotides. A polymerase may be any naturally occurring (i.e., native or wild-type) or engineered variant of a polymerase (e.g., DNA polymerase, Taq polymerase, etc.). Following incorporation of a nucleotide analog comprising a reversible terminator into a nucleic acid strand, the reversible terminator may be removed to permit further extension of the nucleic acid strand. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al.

An alternative design for a probe may be a dual binding probe, or “proximity probe”, which has increased specificity for the target. The proximity probe may include the sequencing primer as part of a second probe with a different target-binding domain, that is specific to a different region/epitope of the same target. In this design, binding of two probes may initiate sequencing, thus increasing specificity because off-target binding may not yield any optical signal. Each probe may comprise 3-4 domains as follows:
(1) A first and second target-related domain. These domains are related to target binding as explained above, with the exception that the two domains target different regions/epitopes in the target molecule;
(2) Primer, one of two variations, or both:
   (a) Primer binding site, to which a sequencing primer can be hybridized;
   (b) Sequencing primer, which can be extended during sequencing;
(3) Flow-based code domain (FCD), as described elsewhere herein; and
(4) Linker between the second target binding domain and the sequencing primer, to minimize steric hindrance and allow binding of the sequencing primer to the primer binding site.

The domains may be spaced with additional linkers composed of either additional nucleotides or other molecules such as PEG. In some cases, part of the primer binding site, the sequencing primer, or both may be designed so that they are folded on another region in the same respective molecule in a secondary structure that reduces the ability of those domains (primer binding site and sequencing primer) to bind each other when the two probes are free. Effective binding may allow the initiation of sequencing only when the two probes are bound in proximity to the same target.

### Base Space Encoding

It will be appreciated that the flow-based code domain as described herein, which distinguishes different probes in the flow space, may be substituted with or used in addition to a base space code domain, which distinguishes different probes in base space. The corresponding sequencing methods and reagents, as well as data analysis, may be selected and used based on which code domain type is used.

For example, each base position may be interrogated by one or more flows that culminate in a unique signal profile that corresponds to a base type at each base position. In some cases, each base position may be interrogated in multiple frequencies or multiple frequency ranges (colors). In some cases, each base position may be interrogated in a single frequency or single frequency range (color). For example, in a multi-color encoding scheme where A is green, C is red, G is both green and red, and T is encoded with no color, the encoding scheme for the probe bases (e.g., “TTT”, “TTA”, etc.) in the base space is as below:
TTT: [0 0 0];
TTA: [0 0 G];
TTC: [0 0 R];
TAT: [0 G 0];
TCT: [0 R 0];
ATT: [G 0 0];
CTT: [R 0 0];
GTT: [G/R 0 0];
Etc.
   where “G” denotes green intensity, “R” denotes red intensity, and “0” denotes background signal that is similar to the 0 flows. In some cases, the interrogating bases (e.g., A, G, C, T/U) may be labeled in 2 colors, 3 colors, 4 colors, or more colors (e.g., red, green, blue, yellow, etc.). The bases may be uniquely labeled by different colors or different combinations of colors, or lack of colors. A “color” as used herein may refer to a distinct frequency or range of frequencies of light that is emitted and detectable, such as upon excitation of a dye that labels a nucleotide base. In some cases, the interrogating bases may be labeled in different intensities in a single color or in multiple colors. For example, a first base (e.g., A) may be labeled with 1 unit of intensity of a first color (such that the interrogation signal translates to ‘1G’ in the base space) and a second base (e.g., C) may be labeled with 2 units of intensity of the first color (such that the interrogation signal translates to ‘2G’ in the base space). For example, this may be achieved via attaching different numbers of the same dye and/or using different dyes detectable at different intensities. In some cases, each base type may be distinguished amongst other bases by a unique signal profile, which may comprise a unique color, unique intensity, or unique combination of frequency and intensity. In some cases, each base position of a code domain may be interrogated by a single flow (e.g., the single flow comprising all 4 bases) or by multiple flows. In some cases, for base space encoding schemes, each entry/element in the base space matrix may correspond to signal(s) representative of a base position. In some cases, for flow-based encoding schemes, each entry/element in the flow space matrix may correspond to signal(s) from a nucleotide flow.

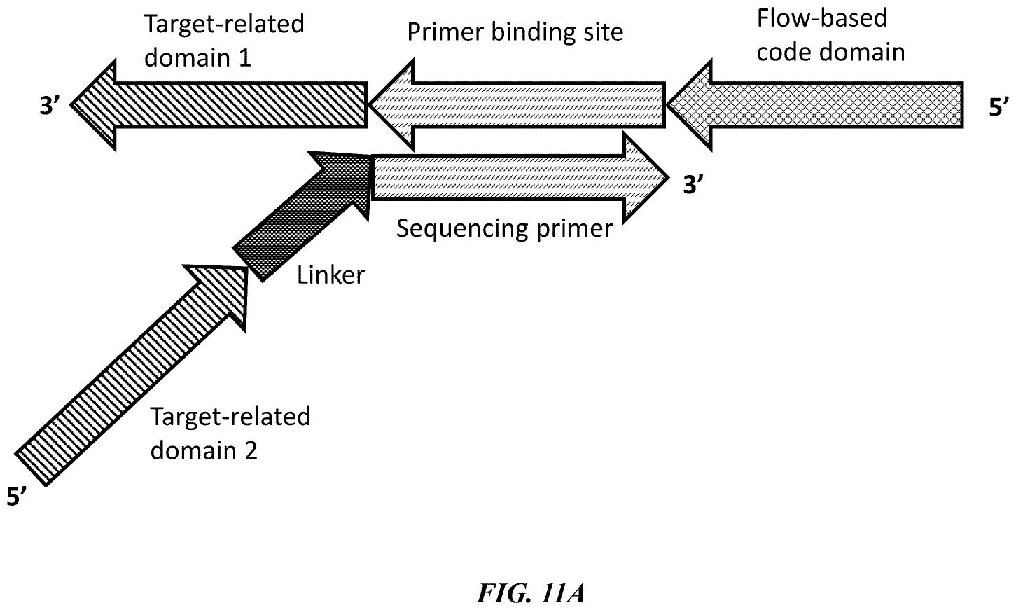

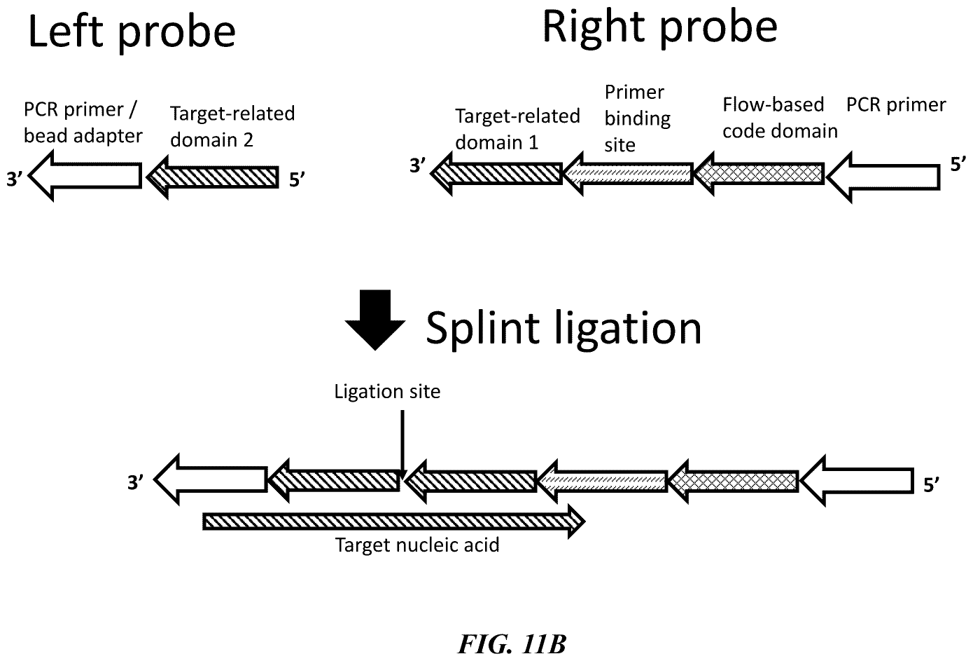

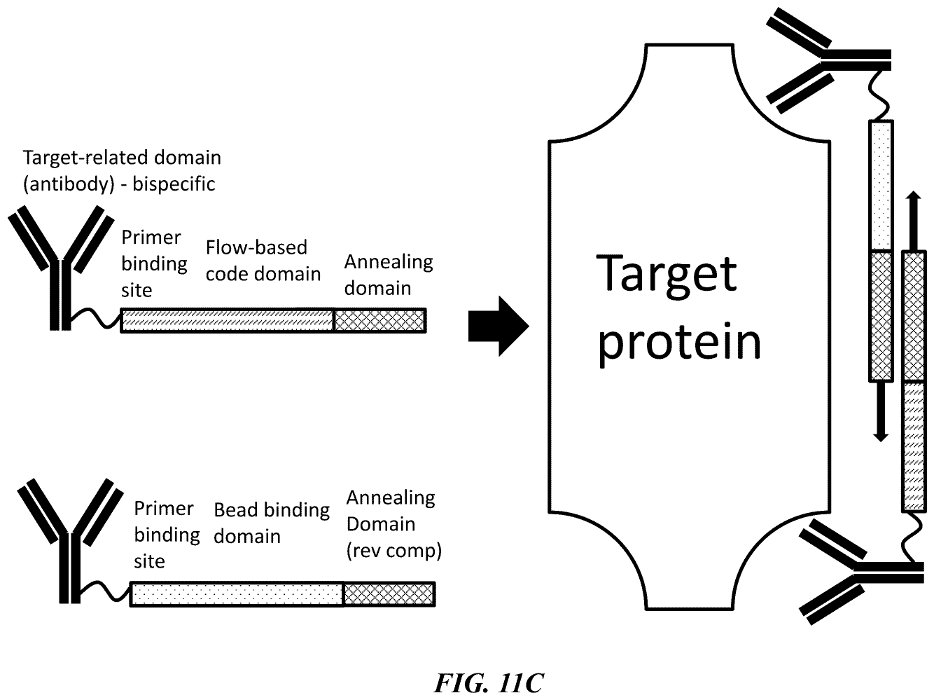

FIGS. 11A-C illustrate exemplary variations of proximity probes that may include a code domain.

FIG. 11A illustrates a two-part probe design that may be used for in-situ sequencing. This probe (which may be referred to as a “proximity probe” or a “proximity probe pair”) comprises two target-related domains that may bind to different proximal locations on the same target analyte. A first strand (shown as the top strand in FIG. 11A) may comprise a first target-related domain (target-related domain 1), a first primer binding site, and a flow-based code domain. A second strand (shown as the bottom strand in FIG. 11A) may comprise a second target-related domain (target-related domain 2), a sequencing primer (which may hybridize to the first primer binding site), and optionally a linker or spacer in between. After the first strand binds to the target analyte via the first target-related domain, the second strand may bind to the target analyte via the second target-related domain while the sequencing primer segment may bind to the first primer binding site. Or vice versa where the second strand binds first and the first strand binds after. In this way, primer annealing and sequencing may be triggered only if the first target site and the second target site (targeted by the first target-related domain and the second target-related domain, respectively) are in proximity. This may greatly increase specificity of the probe for the target analyte.

In some cases, the second target-related domain may be configured to bind to an analyte. In some cases, the analyte may include an antibody or binding fragment thereof, an oligonucleotide, an RNA transcript, a protein, a polypeptide, a metabolite, or genomic DNA. In some cases, the first target-related domain and the second target-related domain may be configured to bind to two different targets. In some cases, the two different targets may be analytes. In some cases, the two different targets may be two different locations on a same molecule. In some cases, the sequencing primer may be hybridized to the primer binding site only when the two different targets are on the same molecule.

FIG. 11B illustrates a two-part probe design that may be used in a splint ligation scheme. This probe (which, similar to the probe illustrated in FIG. 11A, may also be referred to as a proximity probe or proximity probe pair) may comprise two target-related domains (shown as target-related domain 1 and target-related domain 2 in FIG. 11B) that bind to adjacent locations on the same target nucleic acid (e.g., a target transcript). Target-related domain 2 may also be referred to as a “ligation target-related domain”. A first probe part (shown as a “left probe” in FIG. 11B, or also may be referred to as a “3′ probe”) may bind to a first location on the target nucleic acid, and the second probe part (shown as a “right probe” in FIG. 11B, or may be referred to as a “5′ probe”) binds to a second location on the target nucleic acid. The first location and the second location may be adjacent on the target nucleic acid, and the two probe parts may be ligated together. When bound, the first probe part may be disposed 3′ to the second probe, and the second probe may be disposed 5′ to the first probe. Ligated probes may be more stable as compared to non-ligated probes, which may unbind more easily and be washed away. In the scheme shown in FIG. 11B, only ligated probes may be amplified because each probe part contains one PCR primer (one of the PCR primers may be the bead primer, or the bead primer may be added in a consecutive PCR step). In an alternate scheme (not illustrated), two target-related domains may bind to proximal but non-adjacent locations on the same target nucleic acid (e.g., target transcript), where there is a gap in between the non-adjacent locations. After the two probe parts bind to the target nucleic acid, a gap-filling reaction may be conducted to fill in the gap and link the two probe parts together.

In some cases, the probe may be a two-part probe, including a 5′ probe that includes a flow-based code domain comprising a nucleic acid sequence that encodes a flow-space sequence, a first primer binding site, a first target-related domain, and a first PCR primer binding site positioned 5′ to the flow-based code domain; and a 3′ probe comprising a second target-related domain (or ligation target-related domain) and a second PCR binding site. In some cases, the first target-related domain may be configured to bind to an oligonucleotide. In some cases, the second target-related domain (or ligation target-related domain) may be configured to bind to an oligonucleotide. In some cases, the first target-related domain and the second target-related domain (or ligation target-related domain) are configured to bind to adjacent locations on the oligonucleotide. In some cases, the first target-related domain and the ligation target-related domain may be configured to ligate after binding to the oligonucleotide. In some cases, the probe may be bound to a target, wherein a tissue slice comprises the target. In some cases, the probe may be bound to a target that is immobilized on a substrate. In some cases, the substrate may be a Z-slice, a slide, a silicon wafer, or a glass wafer. In some cases, the first target-related domain may bind to a target via a binding agent. In some cases, the binding agent may comprise an oligonucleotide-conjugated antibody.

FIG. 11C illustrates a two-part probe design that may be used in a bispecific antibody binding scheme. This probe (which, similar to the probes illustrated in FIGS. 11A and 11B, may also be referred to as a proximity probe or proximity probe pair) may comprise two antibodies, which respectively may bind to different epitopes of the same target. A first probe part (shown as the top probe in FIG. 11C) may comprise a first antibody conjugated to a first oligonucleotide, the first antibody acting as the target-related domain, and the first oligonucleotide comprising a primer binding site, a flow-based code domain, and a probe annealing domain. The second probe part (shown as the bottom probe in FIG. 11C) may comprise a second antibody conjugated to a second oligonucleotide, the second antibody acting as the target-related domain, and the second oligonucleotide comprising a primer binding site, a bead binding domain, and a probe annealing domain (reverse complement of the probe annealing domain of the first probe part). The first and second antibodies may bind to different, proximal epitopes on the same target (e.g., a target protein). In some cases, the first and second oligonucleotides may bind to each other only when the targets are proximal via the respective probe annealing domains, and thus extend. One or both of the extended strands may then be amplified.

In some cases, the probe may be a two-part probe, comprising a first probe part that comprises a flow-based code domain comprising nucleic acid sequence that encodes a flow-space sequence, a first primer binding site, a first target-related domain, and a first annealing domain positioned 5′ to the flow-based coding domain, wherein the first target-related domain comprises a first antibody; and a second probe part comprising a second target-related domain comprising a second antibody, a second primer binding site, and a second annealing domain, wherein the second annealing domain is configured to be a reverse complement of the first annealing domain. In some cases, the first antibody and the second antibody may bind to two different targets, wherein the two different targets are different locations on a same molecule. In some cases, the second probe part may further comprise a bead-binding domain.

In some cases, the present disclosure provides a plurality of probes or two-part probes of the present disclosure, wherein at least two probes each encodes a unique flow-space sequence.

It will be appreciated that in some instances, the probe of the present disclosure may include numerous variations, changes, and substitutions to the exemplary probes described above. For example, a probe of the present disclosure may include other components to those described above, such as linkers and spacers. In other examples, the order of functional sequences in the probe may be switched. In yet further examples, the probe may include multiple types of sequencing primers and/or primer binding sites, different functional sequences, and the like.

### Preparation and Imaging Protocol

In some cases, binding of the probes to their respective targets may take place by co-incubating them with the sample. Incubation may take place before or after a tissue is placed on a substrate (e.g., wafer), inside or outside of the sequencing apparatus. In some cases, binding is performed in the presence of nonspecific binders in order to reduce background signal, for example a mixture of oligonucleotides or albumin. Binding of the sequencing polymerase and the sequencing primer (if there is a free primer) may take place either in the same incubation with the probes, in a separate reaction following incubation, or within the sequencing apparatus. Alternatively, they may be incubated with the probes before incubation with the sample.

In some cases, in order to reduce secondary structure formation of the probes, various blockers may be used, for example, single strand binding proteins, short random oligonucleotides or short nucleotides complementary to the flow-based coding domain.

In some cases, oligonucleotide probes are extracted from the tissue onto the substrate. Briefly, the oligonucleotide probes containing the flow-based coding domain and sequencing primer are released from the target-related domain after binding, and then the released probes either diffuse towards the substrate and/or are actively extracted using electrophoresis. The substrate itself may be covered by capture oligonucleotides, either directly on its surface or on beads that are immobilized to the substrate. The substrate may also be covered by a conductive material to facilitate electrophoresis.

In some cases, once the probes are captured on the substrate, they may be amplified with RPA, bridge PCR, RCA, MDA and/or another amplification method. In some cases, sequencing may take place on the naked substrate without the tissue.

In some cases, imaging and fluidics apparatus can be either based on a rotating substrate with spin coating, or another imaging system, for example based on a flow cell. Imaging apparatus can either have a low depth of field and scan one or more Z-slices of the sample, or it can have a large depth of field and scan the entire sample simultaneously. In some cases, imaging can be either of single target molecules or of a distribution of many target molecules. The sequencing systems and methods described elsewhere herein may be employed.

In some cases, the fraction of labelled nucleotides in the nucleotide mixture may vary between flows. Specifically, interrogation flows may have a larger bright to natural nucleotide ratio up to 100%, where non-interrogation flows may have lower ratios down to 0. In some cases, imaging in non-interrogation flows is not mandatory.

### Methods

In one aspect, provided herein is a method comprising: (a) binding a probe to a target, the probe comprising: a first flow-based code domain comprising nucleic acid sequence that encodes a flow-space sequence; a first primer binding site; and a first target-related domain configured to bind to the target; (b) hybridizing a sequencing primer to the first primer binding site of the probe; and (c) sequencing at least a portion of the flow-based sequence using flow-based sequencing to generate a flowgram unique to the probe amongst a plurality of probes during flow-based sequencing, wherein the flowgram comprises a set of relative intensity values generated during the flow-based sequencing. In some cases, the method further comprises using the flowgram to determine an identity of the target. In some cases, the target is an analyte, an antibody or fragment thereof, an oligonucleotide, an RNA transcript, a protein, a polypeptide, a metabolite, or genomic DNA. In some cases, the method further includes immobilizing the target on a substrate prior to, during, or subsequent to binding to the probe. In some cases, the method further includes using the flowgram to determine a location and/or distribution of the target on the substrate. In some cases, the substrate may be a Z-slice, a slide, a silicon substrate, or a glass substrate. In some cases, the substrate may comprise a capture oligonucleotide, and the method may further comprise releasing the probe from the target; and binding the probe to the capture oligonucleotide. In some cases, binding the probe to the capture oligonucleotide may be facilitated by electrophoresis, a magnetic field, or a combination thereof. In some cases, the target may be immobilized on a capture bead. In some cases, the probe may be immobilized on a capture bead. In some cases, the probe may comprise more than one flow-based code domain and more than one primer binding site.

In another aspect, provided herein is a method comprising: (a) binding a first probe to a first target, wherein the first probe may include a first flow-based code domain comprising a first nucleic acid sequence that encodes first flow-space sequence; a first primer binding site; and a first target-related domain that is configured to bind to the first target; (b) binding a second probe to a second target, wherein the second probe comprises a second flow-based code domain comprising a second nucleic acid sequence that encodes a second flow-space sequence, a second primer binding site; and a second target-related domain that is configured to bind to the second target; (c) hybridizing a first sequencing primer and a second sequencing primer to the first primer binding site and the second primer binding site, respectively; and (d) sequencing at least a portion of the first flow-space sequence and at least a portion of the second flow-space sequence using flow-based sequencing to generate a first flowgram and a second flowgram, respectively, wherein at least the first flowgram and the second flowgram are unique to each other. In some cases, the first primer binding site and the second primer binding site may comprise an identical sequence. In some cases, the first primer binding site and the second primer binding site comprise different sequences. In some cases, the method may further comprise using the first flowgram and/or the second flowgram to determine an identity of the first target and/or the second target, respectively. In some cases, flow-based sequencing may be performed using between about 1 to about 5000 flows (e.g., nucleotide flows), such as between about 1 to about 4000 flows, about 1 to about 3500 flows, about 1 to about 3000 flows, about 1 to about 2500 flows, about 1 to about 2000 flows, about 1 to about 1500 flows, about 1 to about 1000 flows, about 1 to about 500 flows, about 500 to about 5000 flows, about 500 to about 4500 flows, about 500 to about 4000 flows about 500 to about 3500 flows, about 500 to about 3000 flows, about 500 to about 2500 flows, about 500 to about 2000 flows, about 500 to about 1500 flows about 500 to about 1000 flows, about 1000 to about 5000 flows, about 1000 to about 4500 flows, about 1000 to about 4000 flows, about 1000 to about 3500 flows, about 1000 to about 3000 flows, about 1000 to about 2500 flows, about 1000 to about 2000 flows, about 1000 to about 1500 flows, about 1500 to about 5000 flows, about 1500 to about 4500 flows, about 1500 to about 4000 flows, about 1500 to about 3500 flows, about 1500 to about 3000 flows, about 1500 to about 2500 flows, about 1500 to about 2000 flows, about 2000 to about 5000 flows, about 2000 to about 4500 flows, about 2000 to about 4000 flows about 2000 to about 3500 flows about 2000 to about 3000 flows, about 2000 to about 2500 flows, about 2500 to about 5000 flows about 2500 to about 4500 flows, about 2500 to about 4000 flows, about 2500 to about 3500 flows, about 2500 to about 3000 flows, about 3000 to about 5000 flows, about 3000 to about 4500 flows, about 3000 to about 4000 flows, about 3000 to about 3500 flows, about 3500 to about 5000 flows, about 3500 to about 4500 flows, about 3500 to about 4000 flows, about 4000 to about 5000 flows, or about 4500 to about 5000 flows. In some cases, flow-based sequencing may be performed using about 1, about 5, about 10, about 20, about 50, about 100, about 250, about 500, about 750, about 1000, about 1500, about 2000, about 2500, about 3000, about 3500, about 4000, about 4500, or about 5000 flows. In some cases, flow-based sequencing may be performed with a number of flows that is within a range defined by any two of the preceding values. In some cases, flow-based sequencing may be performed using a set of probes comprising flow-space sequences that are orthogonal to each other in flow space. In some cases, the set of probes may comprise about 1 to about 10⁹ probes, such as between about 1 to about 10⁸ probes, about 1 to about 10⁷ probes, about 1 to about 10⁶ probes, about 1 to about 10⁵ probes, about 1 to about 104 probes, about 1 to about 10³ probes, about 1 to about 10² probes, about 1 to about 10 probes, about 10 to about 10⁹ probes, about 10 to about 10⁸ probes, about 10 to about 10⁷ probes, about 10 to about 10⁶ probes, about 10 to about 10⁵ probes, about 10 to about 10⁴ probes, about 10 to about 10³ probes, about 10 to about 10² probes, about 10² to about 10⁹ probes, about 10² to about 10⁸ probes, about 10² to about 10⁷ probes, about 10² to about 10⁶ probes, about 10² to about 10⁵ probes, about 10² to about 10⁴ probes, about 10² to about 10³ probes, about 10³ to about 10⁹ probes, about 10³ to about 10⁸ probes, about 10³ to about 10⁷ probes, about 10³ to about 10⁶ probes, about 10³ to about 10⁵ probes, about 10³ to about 10⁴ probes, about 10⁴ to about 10⁹ probes, about 10⁴ to about 10⁸ probes, about 10⁴ to about 10⁷ probes, about 10⁴ to about 10⁶ probes, about 10⁴ to about 10⁵ probes, about 10⁵ to about 10⁹ probes, about 10⁵ to about 10⁸ probes, about 10⁵ to about 10⁷ probes, about 10⁵ to about 10⁶ probes, about 10⁶ to about 10⁹ probes, about 10⁶ to about 10⁸ probes, about 10⁶ to about 10⁷ probes, about 10⁷ to about 10⁹ probes, or about 10⁷ to about 10⁸ probes. In some cases, the set of probes may comprise about 1, about 10, about 10², about 10³, about 10⁴, about 10⁵, about 10⁶, about 10⁷, about 10⁸, or about 10⁹ probes. In some cases, the set of probes may comprise a number of probes that is within a range defined by any two of the preceding values. In some cases, the first target and/or the second target may be an analyte. In some cases, the analyte may comprise an antibody or binding fragment thereof, an oligonucleotide, an RNA transcript, a protein, a polypeptide, a metabolite, or genomic DNA. In some cases, the method may further comprise immobilizing the first target and/or second target on a substrate prior to, during, or subsequent to binding the first probe and/or second probe. In some cases, the method may further comprise using the first flowgram and the second flowgram to determine the location and/or distribution of at least the first target and/or second target, respectively, on the substrate. In some cases, the substrate may be a Z-slice, a slide, a silicon substrate, or a glass substrate. In some cases, the first probe and/or the second probe may comprise multiple flow-based code domains and multiple primer binding sites. In some cases, the first probe and/or the second probe may comprise any of the probes or two-part probes as described herein. In some cases, the first target and/or the second target may be from a single cell.

In another aspect, provided herein is a method comprising: (a) immobilizing a first probe and a second probe on a substrate, wherein (i) the first probe may comprise: a first flow-based code domain comprising a first nucleic acid sequence encoding a first flow-space sequence; a first target-related domain that binds to a first target immobilized on the substrate; and a first primer binding site; and (ii) the second probe comprises: a second flow-based code domain comprising a second nucleic acid sequence encoding a second flow-space sequence; a second target-related domain that binds to a second target immobilized on the substrate; and a second primer binding site; (b) hybridizing a first sequencing primer and a second sequencing primer to the first primer binding site and the second primer binding site, respectively; and (c) sequencing at least a portion of the first flow-based code domain and at least a portion of the second flow-based code domain using flow-based sequencing to generate a first flowgram and a second flowgram, respectively, wherein the first flowgram and the second flowgram are unique to each other. In some cases, the first primer binding site and the second primer binding site may comprise an identical sequence. In some cases, the first primer binding site and the second primer binding site comprise different sequences. In some cases, the method may further comprise using the first flowgram and the second flowgram to determine the location and/or distribution of at least the first target and/or second target, respectively, on the substrate. In some cases, the substrate may be a Z-slice, a slide, a silicon substrate, or a glass substrate. In some cases, the first probe and/or the second probe may comprise multiple flow-based code domains and multiple primer binding sites. In some cases, the first probe and/or the second probe may comprise any probe or two-part probe of the present disclosure. In some cases, the first target and/or the second target may be from a single cell.

In another aspect, provided herein is a method comprising: (a) binding a plurality of probes to a plurality of targets, wherein each probe comprises: a flow-based code domain comprising a nucleic acid sequence encoding a flow-space sequence; a primer binding site; and a target-related domain; wherein a plurality of target-related domains binds to the plurality of targets; (b) hybridizing a plurality of sequencing primers to a plurality of primer binding sites; and (c) sequencing a plurality of flow-space sequences of the plurality of probes using flow-based sequencing to generate a plurality of flowgrams, wherein each unique flowgram corresponds to a probe bound to a unique target. In some cases, the method may further include using the plurality of flowgrams to determine an identity of the plurality of targets. In some cases, the plurality of primer binding sites may comprise an identical sequence. In some cases, the plurality of primer binding sites may comprise different sequences. In some cases, the method further comprises immobilizing the plurality of targets on a substrate prior to, during, or subsequent to binding the plurality of probes. In some cases, the method further comprises using the first flowgram and the second flowgram to determine the location and/or distribution of at least the first target and/or second target, respectively, on the substrate. In some cases, the substrate may be a Z-slice, a slide, a silicon substrate, or a glass substrate. In some cases, the plurality of probes may comprise a probe comprising multiple flow-based code domains and multiple primer binding sites. In some cases, the plurality of probes may comprise any probe or two-part probe of the present disclosure. In some cases, the plurality of targets may be from a single cell.

### Systems & Kits

Disclosed herein are systems, kits, and reagents that can be used for or in conjunction with the systems and methods described herein. A kit may comprise any reagent described herein. A system may comprise any kit and/or reagent described herein. A system may comprise a state in which the provided kit has not been used, has been used, or is being used.

For example, a kit may comprise substrates, beads, and/or probes for binding to analytes. A kit may comprise any probe described herein, such as a probe comprising a (1) target-related domain, (2) primer binding site, and (3) flow-based code domain, in any useful order. A kit may comprise any sequencing reagent described herein. A kit may comprise any amplification reagent described herein.

In one example, a kit may include a plurality of probes, each probe comprising: a flow-based code domain comprising a nucleic acid sequence encoding a flow-space sequence, a first primer binding site, and a target-related domain, and instructions for use according to a method of the present disclosure. In some cases, the kit further comprises sequencing reagents, such as single-base nucleotide mixtures (e.g., A, C, G, T or U) or multi-base nucleotide mixtures (e.g., A&C, A&T, A&C&G, etc.). A single-base or multi-base nucleotide mixture may comprise a mixture of labeled and unlabeled nucleotides. A single-base or multi-base nucleotide mixture may comprise non-terminated nucleotides, in some cases comprising only non-terminated nucleotides (vs terminated nucleotides). In some cases, the kit further comprises amplification reagents. In some cases, the kit further comprises a biological sample. In some cases, the biological sample may comprise a tissue. In some cases, the biological sample may be fixed and/or permeabilized. In some cases, the kit further comprises fixing and/or permeabilizing reagents.

In some examples, a system may comprise a sequencing platform configured to perform flow-based sequencing; a plurality of probes, wherein each unique probe comprises a flow-based code domain comprising a nucleic acid sequence encoding a flow-space sequence, a target-related domain and a primer binding site; and a substrate comprising a target. In some cases, the target-related domain is bound to the target. The sequencing platform may be any sequencing platform described herein. The probe may be any probe or two-part probe described herein. The system may further comprise any kit and/or reagent described herein (e.g., beads, indexed data, reagent configured to release oligonucleotide molecules from a plurality of beads, sequencing reagent, amplification reagent, fixing and/or permeabilizing reagent, etc.). In some cases, the system may comprise a light source configured to provide light at desired frequencies (e.g., UV light, fluorescent light, etc.).

### Computer Systems

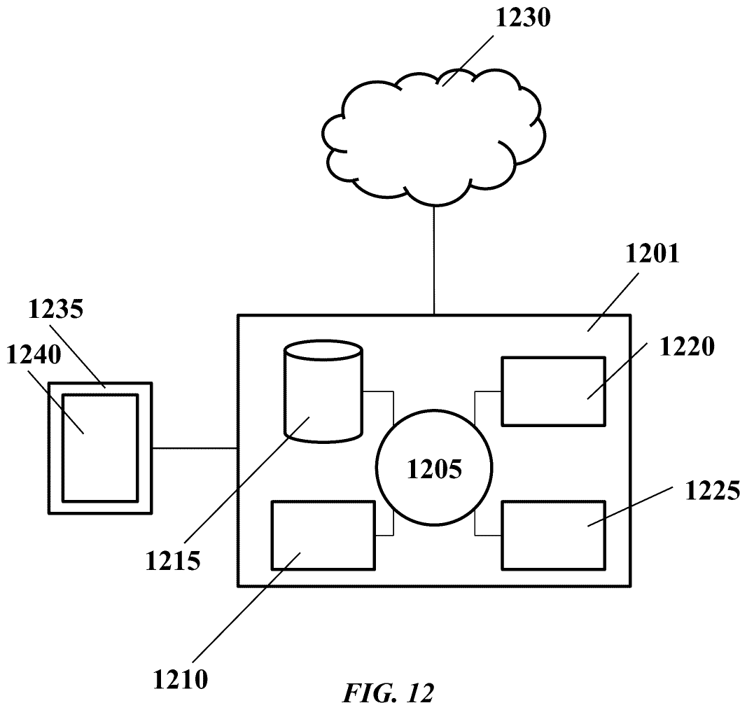

The present disclosure provides computer control systems that are programmed to implement methods of the disclosure. FIG. 12 shows a computer system 1201 that is programmed or otherwise configured to implement methods of the disclosure, such as to control the systems described herein (e.g., reagent dispensing, detecting, etc.) and collect, receive, and/or analyze flow-based sequencing information to conduct spatial analysis of a sample. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an isolated or substantially isolated internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, results of a nucleic acid sequence (e.g., sequence reads), flowgrams, flow-space sequences, and spatial data. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, spatially resolve a plurality of analytes using sequencing information obtained using flow-based sequencing.

### EXAMPLES

### Example 1. In-Situ Workflow Using the Probes Described Herein

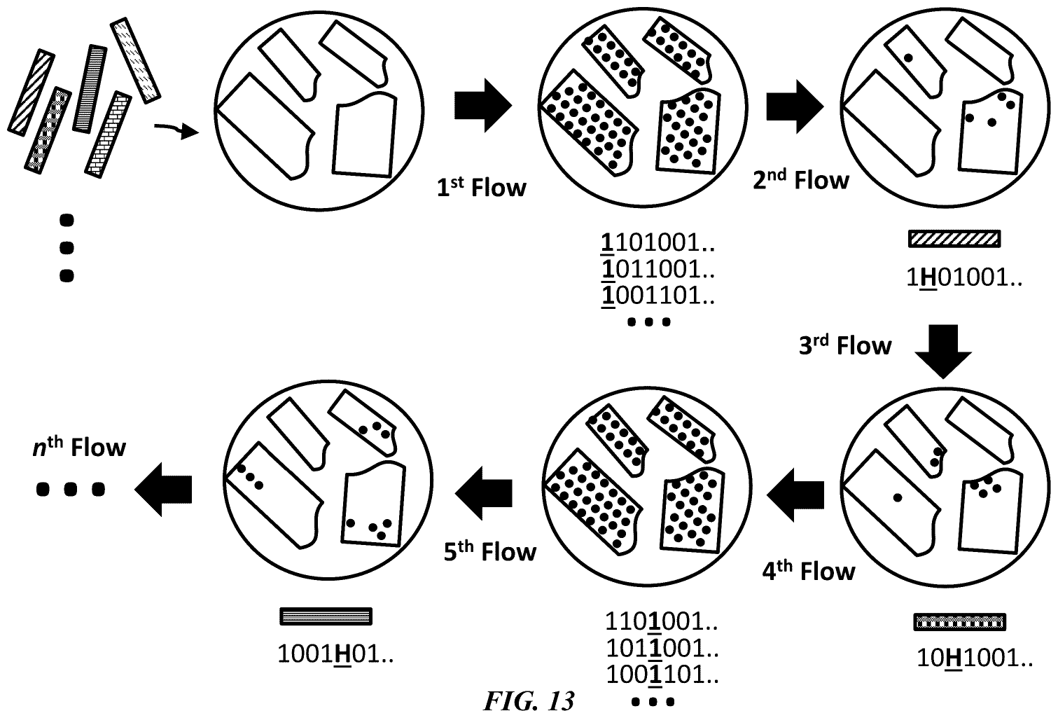

FIG. 13 illustrates an exemplary in-situ workflow using the probes of the present disclosure. The sample (e.g., as tissue slices) is loaded on a substrate (e.g., wafer). Probes are provided to the sample, where they bind to target analytes. Probes can be provided to the sample prior to, during, or subsequent to loading the sample on the substrate. It will be appreciated that the sample may first be incubated with binding agents, which binding agents may be interrogated by the probes. After sufficient incubation to allow probes to bind to their targets, the substrate is subject to flow-based sequencing, as described above. The flow-based code domains in the probes encode the identities of the target analytes at key flow positions (as depicted in FIG. 13 as the 2^{nd} flow, 3^{rd} flow, 5^{th} flow, and so on). If a particular location on the substrate emits a signal (represented as black dots in FIG. 13) at one of the key flow positions, this data may be used to conclude that the tissue slice has a particular target analyte at that particular location. Thus, at each key flow position, the presence or absence of one unique target analyte at specific spatial location may be identified. It will be appreciated that, practically, the signal at any sample location may not be binarily ‘0’ or ‘1’ during the flows, but may be on a more continuous spectrum. For example, most cells will be ‘on’ (emit a non-0 signal) for most flows, with varying intensities.

### Example 2. Probe Extraction Workflow Using the Probes Described Herein

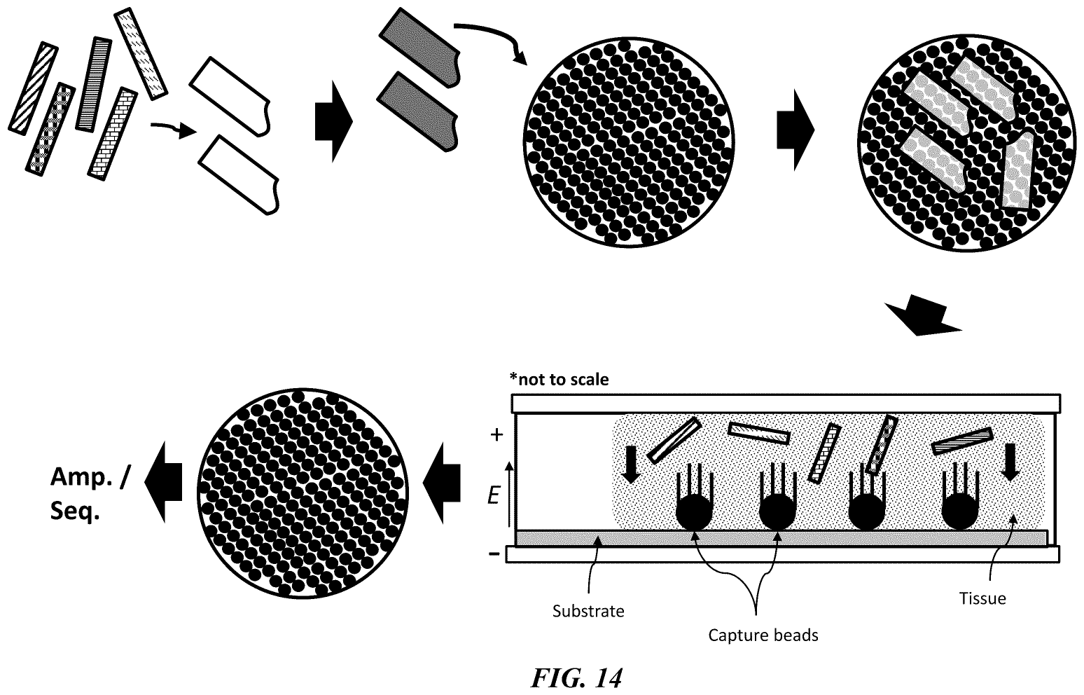

FIG. 14 illustrates an exemplary probe extraction workflow using the probes of the present disclosure. A sample (e.g., tissue slices) is loaded on a substrate (e.g., a wafer). Probes are provided to the sample, where they bind to target analytes. Probes may be provided to the sample prior to, during, or subsequent to loading the sample on the substrate. It will be appreciated that the sample may first be incubated with binding agents, which binding agents may be interrogated by the probes. The substrate is covered with capture oligonucleotides, either directly on the surface or via beads (FIG. 14 illustrates via beads). After sufficient incubation to allow probes to bind to their targets in the sample, the probes or portions thereof are released from the target analytes, such as by releasing (e.g., cleaving) the target-related domain from the remaining portion of the probes. The released probes diffuse towards the wafer and bind to the capture oligonucleotides. Such diffusing can be further facilitated by electrophoresis, where the substrate is sandwiched by conductive material (as illustrated in FIG. 14). In another example, diffusing can be further facilitated by subjecting the space to a magnetic field—e.g., the probes may be bound to magnetic nanoparticles when they are provided to the sample, such that the magnetic nanoparticles can be pulled down with a magnetic field. The sample (e.g., tissue slices) is then removed. Once the probes are captured on the wafer, optionally they are amplified, such as via RPA, bridge PCR, RCA, MDA, or other method. The substrate is subject to flow-based sequencing, as described above. The flow-based code domain in the probes encodes the identities of the target analytes at key flow positions (shown in FIG. 14 as the 2nd flow, 3rd flow, 5th flow, etc.). If a particular location on the substrate emits a signal at one of the key flow positions, this data may be used to conclude that the tissue slice has a particular target analyte at that particular location. Thus, at each key flow position, the presence or absence of one unique target analyte at specific spatial location may be identified.

### Example 3. Polyclonal Bead-Based Workflows Using the Probes Described Herein

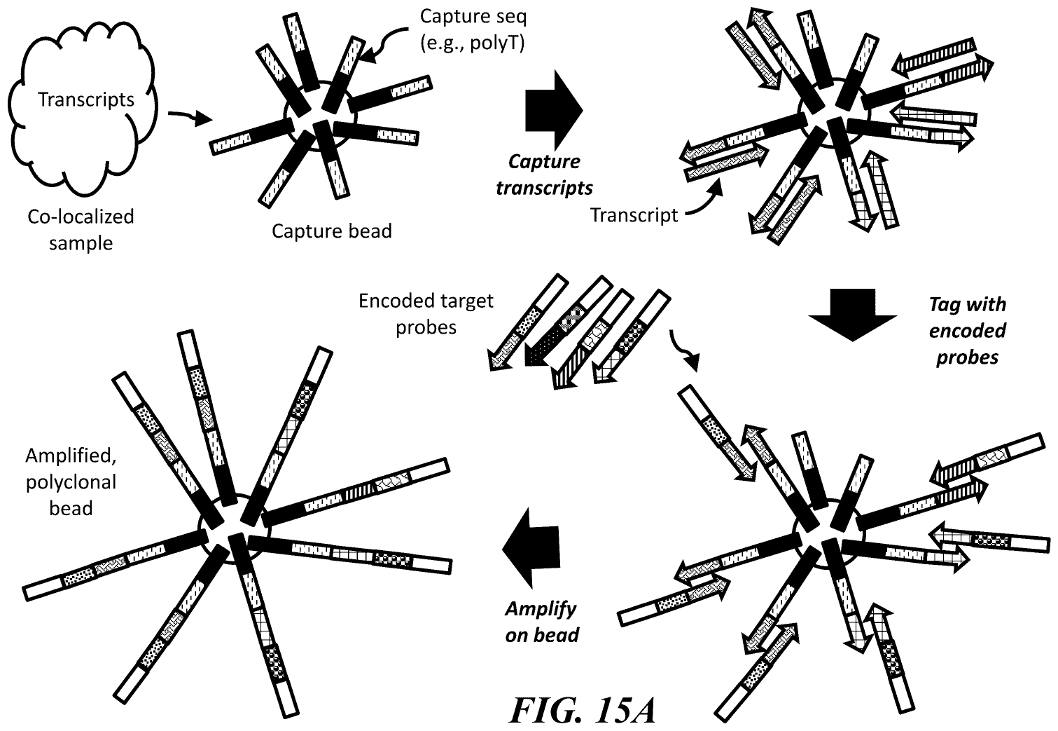

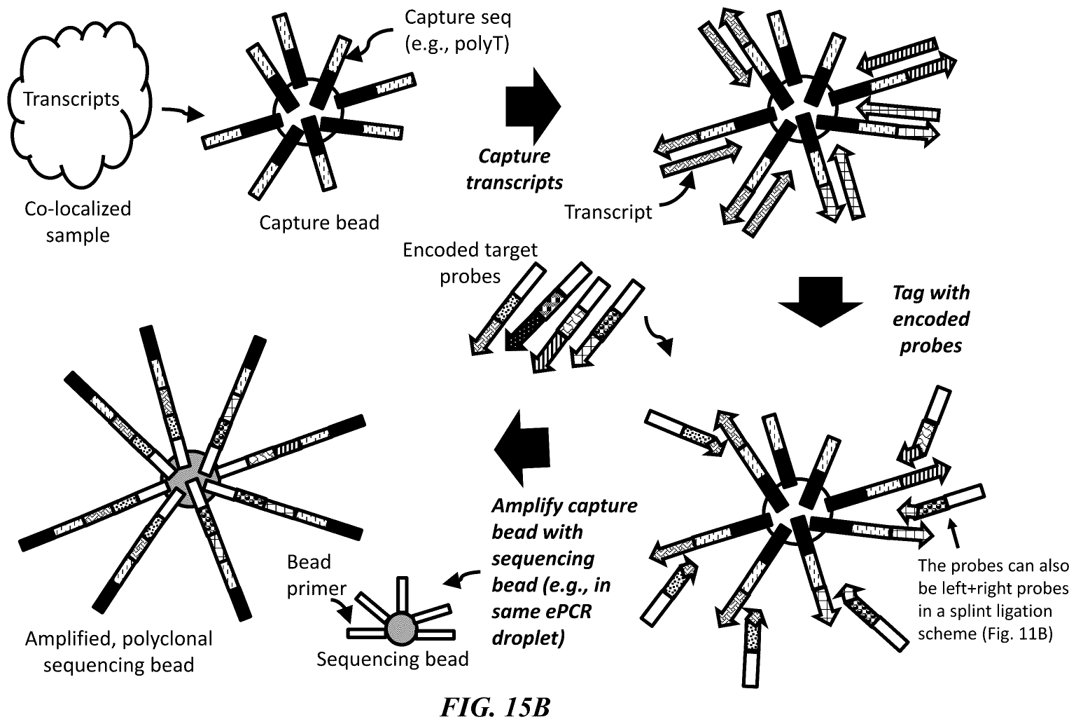

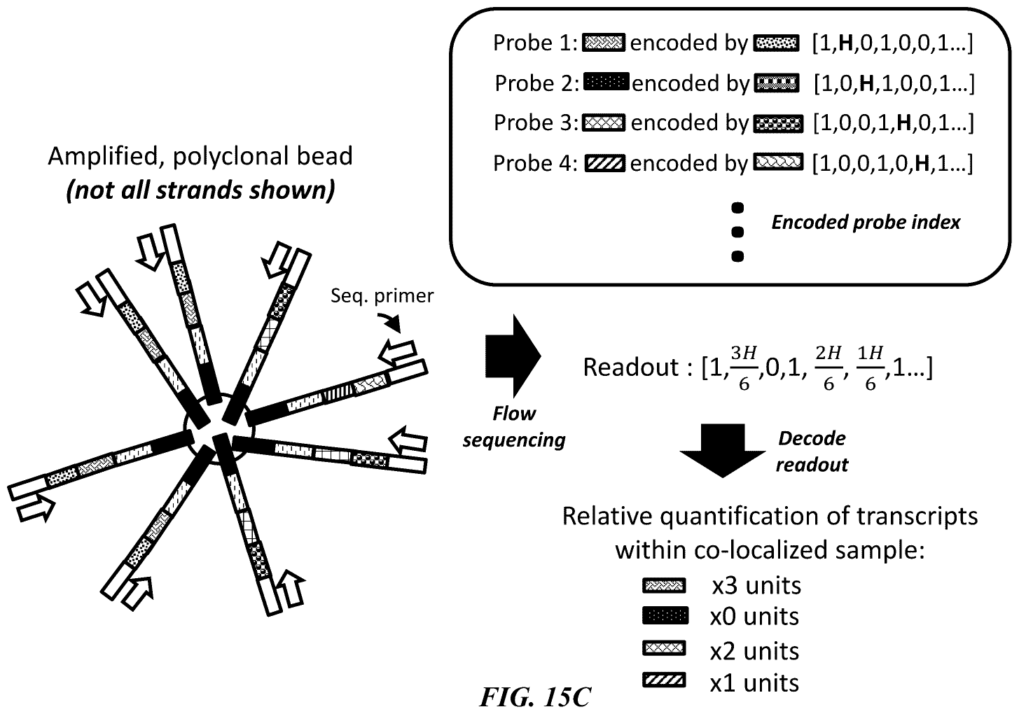

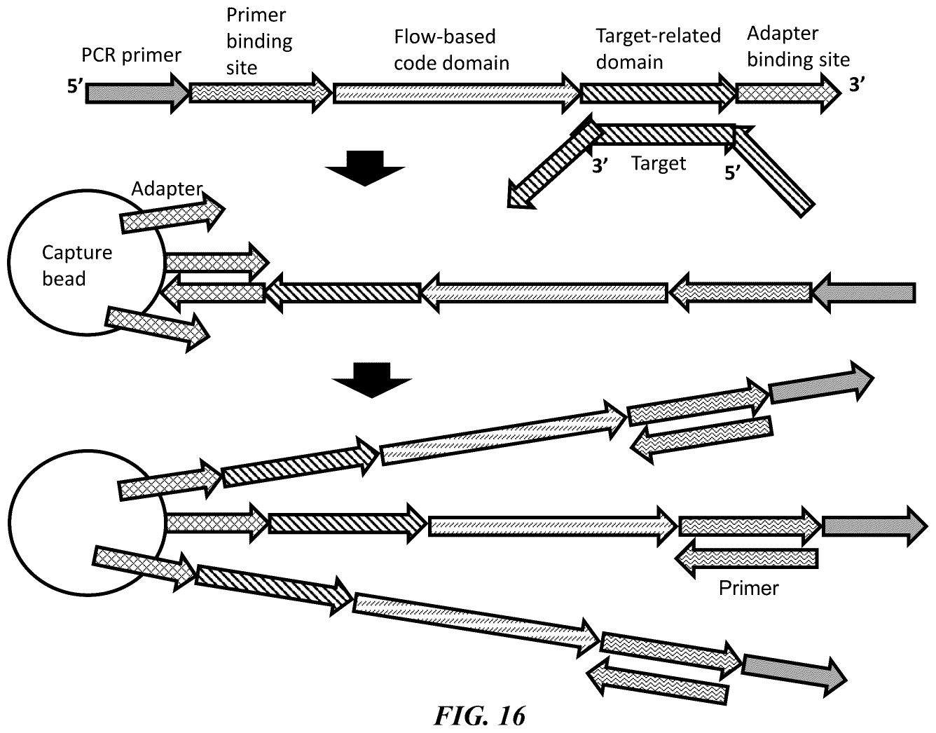

A sample is incubated with capture beads such that analytes from a co-localized sample interact with a single capture bead. In a first workflow (Workflow 1; illustrated in FIG. 15A-B), analytes or analyte proxies are directly captured by a capture bead, and the probes are then provided to the capture bead to interrogate for the analytes or analyte proxies. In a second workflow (Workflow 2; illustrated in FIG. 16), analytes or analyte proxies in the sample are captured by probes, and the probes are then captured by the capture bead.

FIGS. 15A-B illustrate exemplary polyclonal bead-based workflows using the probes of the present disclosure, and in particular, where analytes are localized to a capture bead (Workflow 1). In one example, different co-localized samples (e.g., single cells) and capture beads are partitioned in wells, microwells, or droplets, and render the co-localized sample content accessible to the capture bead. In the single cell context, for example, the cell can be permeabilized and/or lysed. Another example is via the method described with respect to FIG. 14, which relies on diffusion. A substrate is covered with capture beads and a sample is loaded on top of the capture bead array (for example, see FIG. 14), which can be further facilitated by electrophoresis and/or magnetic field to direct the analytes downwards (as opposed to in other directions). Thus, co-localization happens in the z-direction.

A capture bead may comprise a plurality of capture oligonucleotides, each capture oligonucleotide capable of capturing/binding to different analytes. For example, if the target analyte is mRNA, the capture oligonucleotides may comprise a poly-T sequence, which is able to capture different mRNA molecules via the poly-A tail. In another example, where the target analyte of interest is involved in template switching, the capture oligonucleotides can comprise a poly-G (or poly-rG) sequence which is able to capture poly-C containing cDNA (the poly-C sequence is added by the reverse transcription reaction). In another example, the capture oligonucleotides may comprise a random n-mer which may randomly capture different RNA. In another example, which may involve indirect analyte capture, a sample can be incubated first with oligonucleotide-conjugated binding agents (e.g., oligonucleotide-conjugated antibodies) comprising a target sequence and a common sequence, and the capture oligonucleotides can comprise a capture sequence complementary to the common sequence.

After sufficient incubation to allow the targets (or target proxies, such as binding agents or portions thereof) to bind to the capture oligonucleotides, the capture oligonucleotides are extended using the targets or target proxies as templates. The sample, target, and/or target proxies are removed (e.g., by denaturing). The capture beads are then provided with the probes of the present disclosure.

Referring now to FIG. 15A, the probe may comprise the target-related domain, primer binding site, and flow-based code domain. The probe may comprise a different order of the functional sequences than shown in FIG. 10, for example, in order, the primer binding site, the flow-based code domain, and the target-related domain. The target-related domains of the probes may bind to the extended capture oligonucleotides containing the target (or target proxy) sequences. The result will be multiple probes bound to multiple different capture oligonucleotides on the same capture bead. The capture bead may be subjected to amplification, where the different probes bound to the capture bead are amplified, thus producing a polyclonal bead that retains the relative concentrations of the different probes.

Referring now to FIG. 15B, the probe may comprise the target-related domain, flow-based code domain, and bead primer binding site. The probe may comprise the same or different functional sequences than those shown in FIG. 10. The probe may be single part primers. The probe may be two-part primers (e.g., a splint ligation scheme, as shown in FIG. 11B). The target-related domains of the probes may bind to the extended capture oligonucleotides containing the target (or target proxy) sequences. After binding, multiple probes may be bound to multiple different capture oligonucleotides on the same capture bead. Then, the capture bead may be subjected to amplification with a separate sequencing bead. The sequencing bead may comprise a plurality of bead primers. The bead primer may correspond to the bead primer binding site of the probes. The amplification may be performed by, for example, ePCR (e.g., one sequencing bead and one capture bead are contained in the same droplet) or other amplification methods described herein. Only the probes having the correct bead primer binding site may be able to amplify onto the sequencing bead. A polyclonal, amplified sequencing bead may be generated which retains the relative concentrations of the different probes from the capture bead.

Referring now to FIG. 15C, the polyclonal bead may be sequenced by hybridizing a sequencing primer and subjecting it to flow-based sequencing to obtain a flow chart which is informative on the relative concentration. In an example assay where the following probe designs are used:
Probe 1 encoding Target 1: [1 H 0 1 0 0 1 0 0 1 0 0 1 . . . ];
Probe 2 encoding Target 2: [1 0 H 1 0 0 1 0 0 1 0 0 1 . . . ];
Probe 3 encoding Target 3: [1 0 0 1 H 0 1 0 0 1 0 0 1 . . . ];
Probe 4 encoding Target 4: [1 0 0 1 0 H 1 0 0 1 0 0 1 . . . ], and so forth, where each ‘H’ refers to the respective homopolymer length of the probe.

The flow chart for the bead is generated as follows, as illustrated in FIG. 15C:

Readout:

$$[ \begin{matrix}1 & \frac{3 H}{6} & 0 & 1 & \frac{2 H}{6} & \frac{1 H}{6} & 1 & …\end{matrix} ] .$$

The presence or absence of a probe may be determined by the presence or absence of any signal at the key flow position that the probe is encoded at, and the relative concentration of the probe on the polyclonal bead. The relative concentration of the probe on the polyclonal bead represents the relative concentration of the target that the probe codes for in the co-localized sample, and may be determined by the relative intensities of the signals with respect to each other. For example, in the above readout, it may be determined that Targets 1, 3, and 4 are present at the co-localized sample, where there are 3× a unit amount of Target 1, 2× a unit amount of Target 3, and 1× a unit amount of Target 4.

Referring now to FIG. 16, a sample may be incubated with probes to allow probes to bind to target analytes. It will be appreciated that the sample may first be incubated with binding agents, which binding agents may be interrogated by the probes. The probes can contain, from 5′ to 3′, a PCR primer, a primer binding site, a flow-based code domain, a target-related domain, and an adapter binding site. After sufficient incubation to allow probes to bind to their targets or target proxies in the sample, the bound probes can be isolated from the unbound probes. In one example, the sample is fixed (e.g., cells may be fixated by crosslinking the mRNA), the probes are bound to the fixed sample, and any unbound probes are washed away. The isolated bound probes may then be released from the target and downloaded onto the capture bead.

The probes may then be localized to the capture bead. In one example, different co-localized samples (e.g., single cells) and capture beads are partitioned in wells, microwells, or droplets, to render the probes in the co-localized sample accessible to the capture bead. In another example, the probes may be localized to the capture bead via the method described with respect to FIG. 14, which relies on diffusion. A substrate is covered with capture beads and a sample is loaded on top of the capture bead array (for example, see FIG. 14), which can be further facilitated by electrophoresis and/or magnetic field to direct the probes downwards (as opposed to in other directions). Thus, co-localization happens in z-direction. In some cases, the probes or portions thereof are released from the target analytes, such as by releasing (e.g., cleaving) the target-related domain from the remaining portion of the probes.

A capture bead may comprise a plurality of capture oligonucleotides, each capture oligonucleotide comprising an adapter sequence that is complementary to the adapter binding site of the probe. The probe can bind to the capture bead via the adapter binding site. This results in multiple probes bound to multiple different capture oligonucleotides on the same capture bead. The capture bead may be subjected to amplification, where the different probes bound to the capture bead are stochastically amplified, thus producing a polyclonal bead that retains the relative concentrations of the different probes. For simplicity, FIG. 16 shows only one probe bound to the capture bead. The polyclonal bead may be sequenced as described above and illustrated in FIG. 15C.

In some cases, operations may be performed on or off the substrate. For example, amplification may be performed on or off the substrate. When diffusion is used to capture targets or target proxies from the sample on a capture bead-loaded substrate, and amplification is performed on the substrate, spatial relation between different capture beads (for example, between different co-localized samples) may be maintained. If amplification is performed off the substrate, the spatial relation may be maintained within each capture bead (not with other capture beads) in that it is known that all data from the same capture bead is from the same co-localized sample.

### Example 4. Workflow for Single Cell mRNA-Isoform Sequencing Using the Probes Described Herein

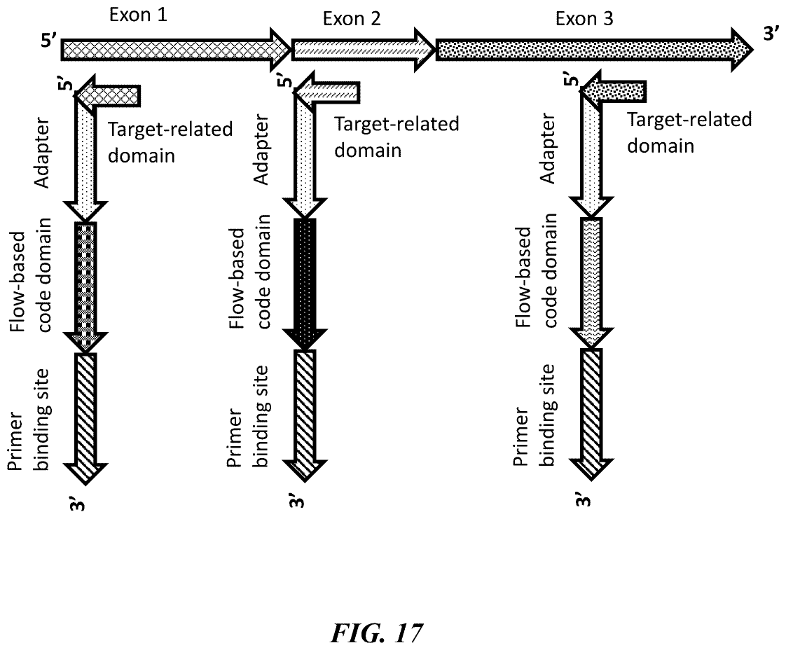

FIG. 17 illustrates an exemplary workflow for single cell mRNA-isoform sequencing using the probes of the present disclosure. In some cases, it may be impractical to read the entire transcript. The workflow shown in FIG. 17 encodes exons with the probes described herein. A probe may be designed to contain, in order, a target-related domain for an exon sequence or a junction between two exons, an adapter sequence, a flow-based code domain, and a primer binding site. The target-related domain may bind to different exons. In some cases, a probe may be designed to include multiple target-related domains per exon or multiple probes may be designed to target the same exon to increase accuracy of the interrogation. Polyclonal beads may be generated by capturing probes from localized samples onto capture beads using the workflow described above and illustrated in FIG. 16.

### Example 5: Workflow for Targeted Ultra-High-Throughput Single Cell Transcriptomics Using the Probes Described Herein

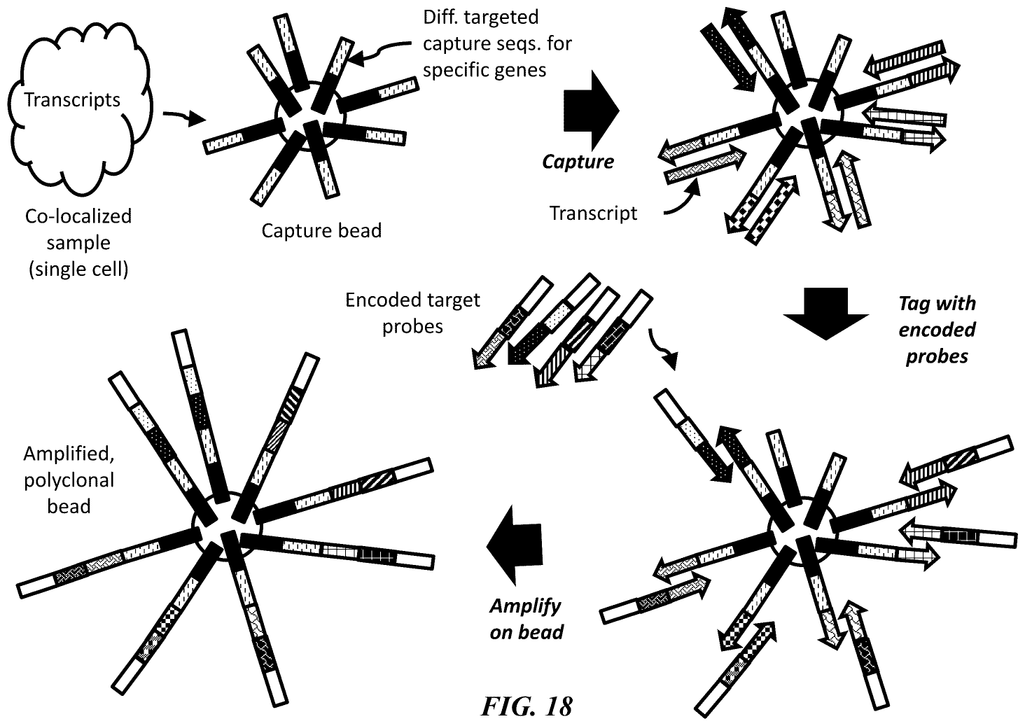

FIG. 18 illustrates an exemplary workflow for targeted ultra-high-throughput single cell transcriptomics using the probes of the present disclosure. This workflow is similar to that illustrated in FIG. 15 in Example 3 above, except that the co-localized sample is a single cell, and the capture bead is covered with different capture oligonucleotides targeting different specific genes.

Various methods may be used to localize analytes to capture beads. In one example, analytes may be localized to capture beads by partitioning different single cells and capture beads in wells, microwells, or droplets, and rendering the single cell accessible to the capture bead, such as via permeabilizing and/or lysing. Another example is via the method described with respect to FIG. 14, which relies on diffusion. For example, a substrate is covered with capture beads and a sample is loaded on top of the capture bead array (e.g., see FIG. 14), which can be further facilitated by electrophoresis to direct the analytes downwards (as opposed to in other directions). In some cases, the sample (e.g., single cells) can be smeared onto the substrate. Thus, co-localization happens in z-direction, and it is very likely that a capture bead captures content only from a single cell.

A capture bead may comprise a plurality of capture oligonucleotides, the capture oligonucleotides configured to capture different genes. After sufficient incubation to allow the targets (or target proxies, such as binding agents or portions thereof) to bind to the capture oligonucleotides, the capture oligonucleotides are extended using the targets or target proxies as templates. The sample, target, and/or target proxies are removed (e.g., by denaturing). The capture beads are then provided with the probes of the present disclosure, which comprises the target-related domain, primer binding site, and flow-based code domain. It will be appreciated that the probe may comprise a different order of the functional sequences other than that shown in FIG. 10, for example, in order, the primer binding site, the flow-based code domain, and the target-related domain. The target-related domains of the probes may bind to the extended capture oligonucleotides containing the target (or target proxy) sequences. This may result in multiple probes bound to multiple different capture oligonucleotides on the same capture bead. The capture bead may be subjected to amplification, and then flow-based sequencing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

The various embodiments described above can be combined to provide further embodiments. Aspects of the embodiments can be modified, if necessary to employ concepts of the various patents, applications and publications mentioned in this specification to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A probe comprising:
a. a flow-based code domain comprising a nucleic acid sequence encoding a flow-space sequence;
b. a first primer binding site; and
c. a first target-related domain.

2. The probe of claim 1, wherein the flow-based code domain is configured to generate a flowgram unique to the probe amongst a plurality of probes during flow-based sequencing, wherein the flowgram comprises a set of relative intensity values generated during the flow-based sequencing.

3. The probe of claim 1 or claim 2, wherein the flow-space sequence comprises a key flow position every (3n−1)^{th} and (3n)^{th} flow positions, wherein n is a positive integer.

4. The probe of any one of claims 1-3, wherein the flow-based code domain is positioned 5′ to the first primer binding site, and the first primer binding site is positioned 5′ to the first target-related domain.

5. The probe of any one of claims 1-4, wherein the first target-related domain comprises an oligonucleotide, an aptamer, an antibody or binding fragment thereof, or a combination thereof.

6. The probe of any one of claims 1-5, wherein the first target-related domain is configured to bind to an analyte.

7. The probe of claim 6, wherein the analyte comprises an antibody or binding fragment thereof, an oligonucleotide, an RNA transcript, a protein, a polypeptide, a metabolite, or genomic DNA.

8. The probe of any one of claims 1-7, wherein the probe further comprises a first PCR primer binding site positioned 5′ to the flow-based code domain or 3′ to the first target-related domain.

9. The probe of any one of claims 1-8, wherein the probe further comprises a bead adapter sequence 3′ to the first target-related domain.

10. The probe of any one of claims 1-8, wherein the probe comprises a first strand and a second strand, wherein the first strand comprises the first target-related domain and the first primer binding site, wherein the second strand comprises a sequencing primer hybridized to the first primer binding site.

11. The probe of claim 10, wherein the second strand further comprises a second target-related domain.

12. The probe of claim 11, wherein the second strand further comprises a linker between the sequencing primer and the second target-related domain.

13. The probe of claim 12, wherein the linker is positioned 5′ to the sequencing primer and the second target-related domain is positioned 5′ to the linker.

14. The probe of any one of claims 11-13, wherein the second target-related domain is configured to bind to an analyte.

15. The probe of claim 14, wherein the analyte comprises an antibody or binding fragment thereof, an oligonucleotide, an RNA transcript, a protein, a polypeptide, a metabolite, or genomic DNA.

16. The probe of any one of claims 11-15, wherein the first target-related domain and the second target-related domain are configured to bind to two different targets.

17. The probe of claim 16, wherein the two different targets are two different locations on a same molecule.

18. A two-part probe, comprising:
a 5′ probe comprising the probe of any one of claims 1-7 and a first PCR primer binding site positioned 5′ to the flow-based code domain; and
a 3′ probe comprising a ligation target-related domain and a second PCR binding site.

19. The two-part probe of claim 18, wherein the first target-related domain is configured to bind to an oligonucleotide.

20. The two-part probe of claim 18 or 19, wherein the ligation target-related domain is configured to bind to an oligonucleotide.

21. The two-part probe of any one of claims 19-20, wherein the first target-related domain and the ligation target-related domain are configured to bind to adjacent locations on the oligonucleotide.

22. The probe or two-part probe of any one of claims 1-21, wherein the probe is bound to a target, wherein a tissue slice comprises the target.

23. The probe or two-part probe of any one of claims 1-22, wherein the probe is bound to a target that is immobilized on a substrate.

24. The probe or two-part probe of claim 23, wherein the substrate is a Z-slice, a slide, a silicon wafer, or a glass wafer.

25. The probe or two-part probe of any one of claims 1-24, wherein the first target-related domain binds to a target via a binding agent.

26. The probe or two-part probe of claim 25, wherein the binding agent comprises an oligonucleotide-conjugated antibody.

27. A two-part probe, comprising:
a first probe part comprising the probe of any one of claims 1-7 and further comprising a first annealing domain positioned 5′ to the flow-based coding domain, wherein the first target-related domain comprises a first antibody; and
a second probe part comprising a second target-related domain comprising a second antibody, a second primer binding site, and a second annealing domain, wherein the second annealing domain is configured to be a reverse complement of the first annealing domain;
wherein the first antibody and the second antibody bind to two different targets, wherein the two different targets are different locations on a same molecule.

28. The two-part probe of claim 27, wherein the second probe part further comprises a bead-binding domain.

29. A plurality of the probes or two-part probes of any one of claims 1-28, wherein at least two probes each encode a unique flow-space sequence.

30. A method comprising:
a. binding a probe to a target, the probe comprising:
a first flow-based code domain comprising a nucleic acid sequence encoding a flow-space sequence;
a first primer binding site; and
a first target-related domain configured to bind to the target;
b. hybridizing a sequencing primer to the first primer binding site of the probe; and
c. sequencing at least a portion of the flow-based sequence using flow-based sequencing to generate a flowgram unique to the probe amongst a plurality of probes during flow-based sequencing, wherein the flowgram comprises a set of relative intensity values generated during the flow-based sequencing.

31. The method of claim 30, further comprising using the flowgram to determine an identity of the target.

32. The method of claim 30 or claim 31, wherein the target is an analyte, an antibody or fragment thereof, an oligonucleotide, an RNA transcript, a protein, a polypeptide, a metabolite, or genomic DNA.

33. The method of any one of claims 30-32, further comprising immobilizing the target on a substrate prior to, during, or subsequent to binding to the probe.

34. The method of claim 33, further comprising using the flowgram to determine a location and/or distribution of the target on the substrate.

35. The method of claim 33 or claim 34, wherein the substrate is a Z-slice, a slide, a silicon wafer, or a glass wafer.

36. The method of any one of claims 33-35, wherein the substrate further comprises a capture oligonucleotide, and the method further comprises:
releasing the probe from the target; and
binding the probe to the capture oligonucleotide.

37. The method of claim 36, wherein binding the probe to the capture oligonucleotide is facilitated by electrophoresis, a magnetic field, or a combination thereof.

38. The method of any one of claims 30-37, wherein the target is immobilized on a capture bead.

39. The method of any one of claims 30-37, wherein the probe is immobilized on a capture bead.

40. The method of any one of claims 30-39, wherein the probe comprises more than one flow-based code domain and more than one primer binding site.

41. The method of any one of claims 30-39, wherein the probe comprises the probe or two-part probe of any one of claims 1-28.

42. A method comprising:
a. binding a first probe to a first target, wherein the first probe comprises:
a first flow-based code domain comprising a first nucleic acid sequence encoding a first flow-space sequence;
a first primer binding site; and
a first target-related domain that is configured to bind to the first target;
b. binding a second probe to a second target, wherein the second probe comprises:
a second flow-based code domain comprising a second nucleic acid sequence encoding a second flow-space sequence;
a second primer binding site; and
a second target-related domain that is configured to bind to the second target;
c. hybridizing a first sequencing primer and a second sequencing primer to the first primer binding site and the second primer binding site, respectively; and
d. sequencing a portion of the first flow-space sequence and a portion of the second flow-space sequence using flow-based sequencing to generate a first flowgram and a second flowgram, respectively, wherein the first flowgram and the second flowgram are unique to each other.

43. The method of claim 42, wherein the first primer binding site and the second primer binding site comprise an identical sequence.

44. The method of claim 42, wherein the first primer binding site and the second primer binding site comprise different sequences.

45. The method of any one of claims 42-44, further comprising using the first flowgram and/or the second flowgram to determine an identity of the first target and/or the second target, respectively.

46. The method of any one of claims 42-45, wherein the first target and/or the second target is an analyte.

47. The method of claim 46, wherein the analyte comprises an antibody or binding fragment thereof, an oligonucleotide, an RNA transcript, a protein, a polypeptide, a metabolite, or genomic DNA.

48. The method of any one of claims 42-47, further comprising immobilizing the first target and/or second target on a substrate prior to, during, or subsequent to binding the first probe and/or second probe.

49. The method of claim 48, further comprising using the first flowgram and the second flowgram to determine the location and/or distribution of at least the first target and/or second target, respectively, on the substrate.

50. The method of claim 48 or claim 49, wherein the substrate is a Z-slice, a slide, a silicon wafer, or a glass wafer.

51. The method of any one of claims 42-50, wherein the first probe and/or the second probe comprises multiple flow-based code domains and multiple primer binding sites.

52. The method of any one of claims 42-50, wherein the first probe and/or the second probe comprise the probe or two-part probe of any one of claims 1-28.

53. The method of any one of claims 42-52, wherein the first target and/or the second target are from a single cell.

54. A method comprising:
a. immobilizing a first probe and a second probe on a substrate, wherein:
the first probe comprises:
a first flow-based code domain comprising a first nucleic acid sequence encoding a first flow-space sequence;
a first target-related domain that binds to a first target immobilized on the substrate; and
a first primer binding site; and
the second probe comprises:
a second flow-based code domain comprising a second nucleic acid sequence encoding a second flow-space sequence;
a second target-related domain that binds to a second target immobilized on the substrate; and
a second primer binding site;
b. hybridizing a first sequencing primer and a second sequencing primer to the first primer binding site and the second primer binding site, respectively; and
c. sequencing a portion of the first flow-based code domain and a portion of the second flow-based code domain using flow-based sequencing to generate a first flowgram and a second flowgram, respectively, wherein the first flowgram and the second flowgram are unique to each other.

55. The method of claim 54, wherein the first primer binding site and the second primer binding site comprise an identical sequence.

56. The method of claim 54, wherein the first primer binding site and the second primer binding site comprise different sequences.

57. The method of any one of claims 54-56, further comprising using the first flowgram and the second flowgram to determine the location and/or distribution of at least the first target and/or second target, respectively, on the substrate.

58. The method of any one of claims 54-57, wherein the substrate is a Z-slice, a slide, a silicon wafer, or a glass wafer.

59. The method of any one of claims 54-58, wherein the first probe and/or the second probe comprises multiple flow-based code domains and multiple primer binding sites.

60. The method of any one of claims 54-58, wherein the first probe and/or the second probe comprise the probe or two-part probe of any one of claims 1-28.

61. The method of any one of claims 54-60, wherein the first target and/or the second target are from a single cell.

62. A method comprising:
a. binding a plurality of probes to a plurality of targets, wherein each probe comprises:
a flow-based code domain comprising a nucleic acid sequence encoding a flow-space sequence;
a primer binding site; and
a target-related domain;
wherein a plurality of target-related domains binds to the plurality of targets;
b. hybridizing a plurality of sequencing primers to a plurality of primer binding sites; and
c. sequencing a plurality of flow-space sequences of the plurality of probes using flow-based sequencing to generate a plurality of flowgrams, wherein each unique flowgram corresponds to a probe bound to a unique target.

63. The method of claim 62, further comprising using the plurality of flowgrams to determine an identity of the plurality of targets.

64. The method of claim 62 or claim 63, wherein the plurality of primer binding sites comprises an identical sequence.

65. The method of claim 62 or claim 63, wherein the plurality of primer binding sites comprises different sequences.

66. The method of any one of claims 62-65, further comprising immobilizing the plurality of targets on a substrate prior to, during, or subsequent to binding the plurality of probes.

67. The method of claim 66, further comprising using the first flowgram and the second flowgram to determine the location and/or distribution of at least the first target and/or second target, respectively, on the substrate.

68. The method of claim 66 or claim 67, wherein the substrate is a Z-slice, a slide, a silicon wafer, or a glass wafer.

69. The method of any one of claims 62-68, wherein the plurality of probes comprises a probe comprising multiple flow-based code domains and multiple primer binding sites.

70. The method of any one of claims 62-68, wherein the plurality of probes comprises the probe or two-part probe of any one of claims 1-28.

71. The method of any one of claims 62-70, wherein the plurality of targets is from a single cell.

72. A system comprising:
a sequencing platform configured to perform flow-based sequencing;
a plurality of probes, wherein each unique probe comprises a flow-based code domain comprising a nucleic acid sequence encoding a flow-space sequence, a target-related domain and a primer binding site; and
a substrate comprising a target, wherein the target-related domain is bound to the target.

73. The system of claim 72, wherein the plurality of probes comprises the probe or two-part probe of any one of claims 1-28.

74. A kit comprising:
a plurality of probes, each probe comprising:
a flow-based code domain comprising a nucleic acid sequence encoding a flow-space sequence;
a first primer binding site; and
a target-related domain; and
instructions for use according to any one of the methods of claims 30-71.


SEQUENCE LISTING (2 sequences)

SEQ ID NO: 1 - DNA, 25 bp, synthetic construct
  source 1..25: mol_type = other DNA
atgcatgcatgatgatgatgcatgc

SEQ ID NO: 2 - DNA, 12 bp, synthetic construct
  source 1..12: mol_type = other DNA
tatggtcgtcga